(19) United States
(12) Patent Application Publication
(10) Pub. No.: US 2026/0258361 A1
(43) Pub. Date: Sep. 3, 2026

(54) METHOD FOR ISOLATING HEMOGENIC ENDOTHELIAL CELLS

(71) Applicants: FONDAZIONE TELETHON ETS, Rome (IT); OSPEDALE SAN RAFFAELE S.R.L., Milan (IT)

(72) Inventors: Andrea DITADI, Milan (IT); Rebecca SCARFO', Milan (IT)

(73) Assignees: FONDAZIONE TELETHON ETS, Rome (IT); OSPEDALE SAN RAFFAELE S.R.L., Milan (IT)

(21) Appl. No.: 18/877,618

(22) PCT Filed: Jun. 22, 2023
(86) PCT No.: PCT/EP2023/067025

§ 371 Date: Dec. 20, 2024

(30) Foreign Application Priority Data
Jun. 22, 2022 (EP) 22180407.3

(51) Int. Cl. C12N 5/0789; A61K 35/28

(52) U.S. Cl. CPC C12N 5/0647; A61K 35/28; C12N 2501/105; C12N 2501/115; C12N 2501/125; C12N 2501/14; C12N 2501/155; C12N 2501/165; C12N 2501/2306; C12N 2501/2311; C12N 2501/415; C12N 2506/02; C12N 2506/45

(57) ABSTRACT

The present invention refers to An in vitro method of isolating hemogenic endothelial cells (HECs) or enriched populations of HECs, comprising isolating CD32+ cells from a population of cells derived from pluripotent stem cells.

### TECHNICAL FIELD

The present invention refers to an in vitro method of isolating hemogenic endothelial cells (HECs) or enriched populations of HECs, comprising isolating CD32+ cells from a population of cells derived from pluripotent stem cells, or of generating a population of hematopoietic cells from said HECs and to cells or populations obtainable by said methods and to uses thereof.

### BACKGROUND ART

Throughout life, hematopoiesis, i.e., blood cell production, is guaranteed by hematopoietic stem cells (HSCs). For their regenerative capacity, HSCs have been extensively exploited as a routine treatment for blood disorders and malignant diseases (Orkin & Zon, 2008). In addition to HSCs, the transfusion of mature blood cells is an effective therapeutic treatment in many conditions. Understanding the molecular regulation of blood cell formation during embryogenesis is critical for the development of efficient protocols to generate hematopoietic cells in vitro, in particular from human pluripotent stem cells (hPSCs). Various experimental models have informed us that, during development, hematopoietic cells emerge from hemogenic endothelial cells (HECs), a specialized subpopulation of embryonic endothelium (Jaffredo et al, 1998; Zovein et al, 2008; Bertrand et al, 2010; Boisset et al, 2010; DeBruijn et al, 2000). HECs generate blood cells via an endothelial-to-hematopoietic transition (EHT), which involves considerable transcriptional and morphological changes leading to the identity switch from endothelial cell to blood (Jaffredo et al, 1998; DeBruijn et al, 2000; Zovein et al, 2008; Kissa & Herbomel, 2010; Boisset et al, 2010; Bertrand et al, 2010). HECs is a central element of most of, if not all, the successive spatio-temporal regulated waves of embryonic hematopoiesis that produce blood progenitors with distinct potential and function (Zovein et al, 2008; Lancrin et al, 2009; McGrath et al, 2015; Frame et al, 2016; Stefanska et al, 2017). For this reason, HECs that can be found in different anatomical locations (Swiers et al, 2013b), the best characterized of which are the yolk sac (YS) and the aorta-gonad-mesonephros (AGM), where the development of hematopoietic clusters (HCs) budding from HECs within the lumen of arteries is observed (Dzierzak & Bigas, 2018). As HECs represent a small and transient population rapidly undergoing EHT, they have been difficult to study and characterize. In particular, while the endothelial descendancy of blood cells is well established, the identity of HECs is still debated. As such, the heterogeneity of HECs harboring distinct hematopoietic potential has remained unresolved, hampering the design of accurate protocols for the derivation of specific hematopoietic cells from hPSCs. Traditionally, HECs have been isolated using reporters under the control of the regulatory elements of transcription factors that drive EHT, such as Runx1 (North et al, 1999; Swiers et al, 2013a; Thambyrajah et al, 2016). This strategy cannot be used for the isolation of HECs from human embryos and the introduction of a reporter vector into an hPSC line would prevent their clinical application, as it would result in genetically modified blood cells. Recently, transcriptomic analyses have allowed the identification of putative HEC markers in both murine and human embryos (Zeng et al, 2019; Oatley et al, 2020; Fadlullah et al, 2021; Dignum et al, 2021), which however also enrich for arterial endothelial cells that are associated with HECs (Robert-Moreno et al, 2008; Yamamizu et al, 2010; Ditadi et al, 2015). This hinders the specific characterization of the unique endothelial population that generate blood cells.

Therefore, there is still the need for a method for efficiently isolate HECs.

### SUMMARY OF THE INVENTION

During embryonic development, blood cells emerge from a subset of specialized endothelial cells, named hemogenic endothelial cells (HECs), via a process known as endothelial-to-hematopoietic transition (EHT). A more thorough characterization of HECs is essential to guide the efforts to derive this population from human pluripotent stem cells (hPSCs), a critical step to generate therapeutic blood products in vitro. However, current known markers used to isolate HECs are insufficient as they also enrich for arterial endothelial cells that are associated with HECs. To identify specific human HEC markers, inventors observed that the expression of the Fc receptor CD32 previously associated with other specialized endothelia, including early hematopoietic progenitors expressing endothelial markers in the mouse embryo, demarcates a subset of CD34⁺CD73^{neg}CD184^{neg}DLL4^{neg} endothelial cell population that contains HECs. The inventors also observed that CD32 is expressed in the human embryos. Functional ex vivo analyses confirmed that multilineage hematopoietic potential is highly enriched in CD32+ endothelial cells isolated from the AGM and YS of human embryos. CD32 emerged as selective marker also for hPSC-derived HECs across different hematopoietic programs. Remarkably, the present analyses showed that CD32 expression enriches for cells with hemogenic potential with a higher specificity for hPSC-derived HECs than other known HEC markers. These findings provide a simple method for isolating HECs from hPSC cultures, allowing its molecular characterization as well as the efficient generation of hematopoietic cells in vitro.

An object of the present invention is an in vitro method of isolating hemogenic endothelial cells (HECs) or enriched populations of HECs, comprising isolating CD32+ cells from a population of cells derived from pluripotent stem cells.

Preferably the method comprises the steps of:
i. Culturing a population of pluripotent stem cells (PSCs) in a mesoderm differentiation medium and/or hematovascular differentiation medium;
ii. Isolating CD32+ cells from the population of cells obtained from step (i) to obtain hemogenic endothelial cells (HECs) or enriched populations of HECs.

Therefore an object of the invention is also an in vitro method of isolating hemogenic endothelial cells (HECs) or enriched populations of HECs comprising the steps of:
i. Culturing a population of pluripotent stem cells (PSCs) (or PSCs) in a mesoderm differentiation medium and/or hematovascular differentiation medium;
ii. Isolating CD32+ cells from the population of cells (or from the cells) obtained from step (i) to obtain hemogenic endothelial cells (HECs) or enriched populations of HECs.

Preferably, the HECs are capable of generating hematopoietic cells in vitro or following transplantation into a recipient.

Preferably, the HECs are increased in numbers and percentage in cell population and/or have increased potency in differentiation and/or improved HE cellularity, as compared to culturing pluripotent stem cells having HECs not previously isolated for the presence of CD32+.

Another object of the invention is an in vitro method of generating a population of hematopoietic cells comprising the steps of:
i. Culturing a population of pluripotent stem cell (PSC) (or PSCs) in a mesoderm differentiation medium and/or hematovascular differentiation medium;
ii. Isolating CD32+ cells from the population of cells (or from cells) obtained from step (i) to obtain hemogenic endothelial cells (HECs) or enriched populations of HECs,
iii. culturing the obtained hemogenic endothelial cells (HECs) or enriched populations of HECs obtained from step (ii) in a medium that induces or promote hematopoietic cells, preferably a hematopoietic specification medium, to produce a population of hematopoietic cells (or HCs).

Preferably the pluripotent stem cells are derived embryonic stem cells or ESCs or induced pluripotent stem cells (iPSCs), preferably human, and/or the pluripotent stem cells (PSCs) are derived from cultures or lines or tissues.

Preferably the isolation step further comprises selection of cells based on expression of at least one additional endothelial marker and/or at least one marker selected from CD34, CD31, CD54, CD102, CD47, CD144, CD146, CD124, CD201, CD151, LTBR, CD39, CD147, CD105, CD143, CD309, CD44.

Preferably the isolation step further comprises selection of cells that do not express at least one marker selected from CD43, CD184, CD73 and/or DLL4.

Preferably the cells are isolated (and/or selected) by an immunoselection technique, for example selected from the group consisting of flow cytometry, activated cells sorting and immune-magnetic selection, and/or by means of a specific ligand, preferably the ligand is an antibody, or functional fragment thereof, preferably by using a carrier conjugated to said ligand, or by FACS, preferably wherein the carrier is a magnetic bead or wherein the carrier is loaded into a column.

Another object of the invention are isolated HECs or the enriched populations of HECs obtainable by the method as disclose herein or above or the population of hematopoietic cells obtainable by the method as disclosed herein or above.

Another object of the invention is a substantially pure population of CD32+ HECs or a population of hematopoietic cells derived from a substantially pure population of CD32+ HECs.

A further object of the invention is an isolated HECs or the enriched populations of HECs as defined herein or the population of hematopoietic cells as defined herein or the substantially pure population of CD32+ HECs as defined herein or the population of hematopoietic cells derived from a substantially pure population of CD32+ HECs as defined herein or cells derived therefrom wherein the cells or the population is CD32+CD34+CD43−CD184−CD73−DLL4− and/or wherein the population comprises at least 80% or 85% or 90% of hematopoietic cells.

Another object of the invention is a composition comprising the isolated HECs or the enriched populations of HECs as defined herein or the population of hematopoietic cells as defined herein or the substantially pure population of CD32+ HECs as defined herein or the population of hematopoietic cells derived from a substantially pure population of CD32+ HECs as defined herein or cells derived therefrom, preferably wherein said composition further comprises a pharmaceutically acceptable carrier and/or a culture medium and/or a cryoprotective agent.

A further object of the invention is the population of hematopoietic cells as defined herein or the population of hematopoietic cells derived from a substantially pure population of CD32+ HECs as defined herein or the composition comprising the population of hematopoietic cells or the population of hematopoietic cells derived from a substantially pure population of CD32+HEC as defined herein or cells derived therefrom for medical use, preferably for use in the prevention and/or treatment of hematopoietic disorders and diseases, for example resulting from a hematopoietic malignancy, an immunodeficiency disorder, preferably wherein said immunodeficiency disorder is selected from a T-cell deficiency, a B-cell deficiency, a combined T-cell/B-cell deficiency, an antibody deficiency, a complement deficiency, leukemia, lymphoma, anemia, neutropenia, lymphopenia, lupus, and Wiskott-Aldrich syndrome, preferably wherein said immunodeficiency disorder results from administration of an immunosuppressive or cytotoxic agent or from infection with human immunodeficiency virus (HIV) or hepatitis, or of cancer, or for cell and gene therapy, e.g. for treatment of genetic disorders or cancer or infections, or for regenerative medicine, or for use as blood cells for transfusions.

Another object of the invention is the use of a ligand specific for CD32 for the detection and/or identification and/or isolation of HECs in a population derived from pluripotent stem cells, preferably wherein the ligand is an anti-CD 32 antibody, or functional fragments thereof.

A further object of the invention is a kit comprising detecting means for CD32 and/or at least one of the markers as defined herein for carrying out the method as defined herein.

The invention will be illustrated by means of non-limiting examples in reference to the following figures.

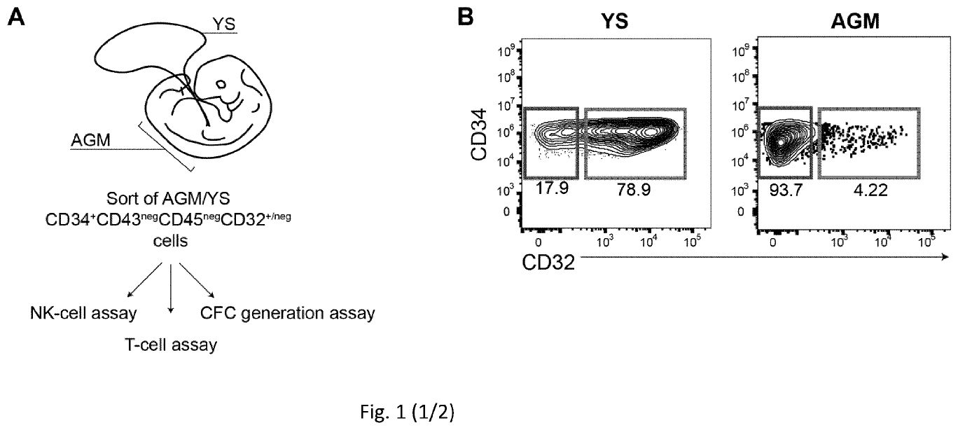

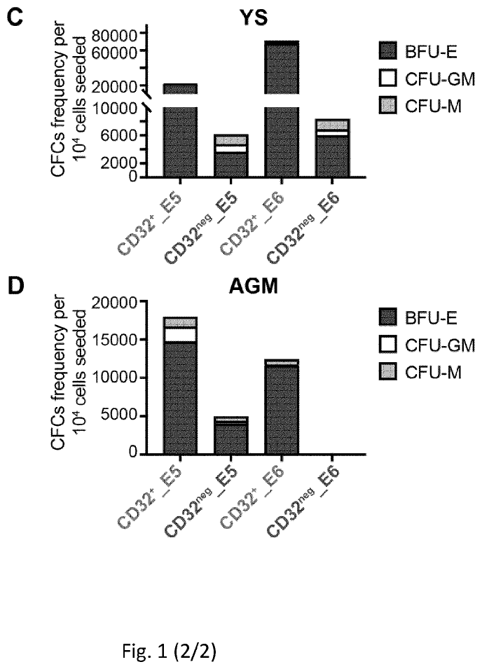

FIG. 1: CD32 is expressed in HECs during human embryonic development.

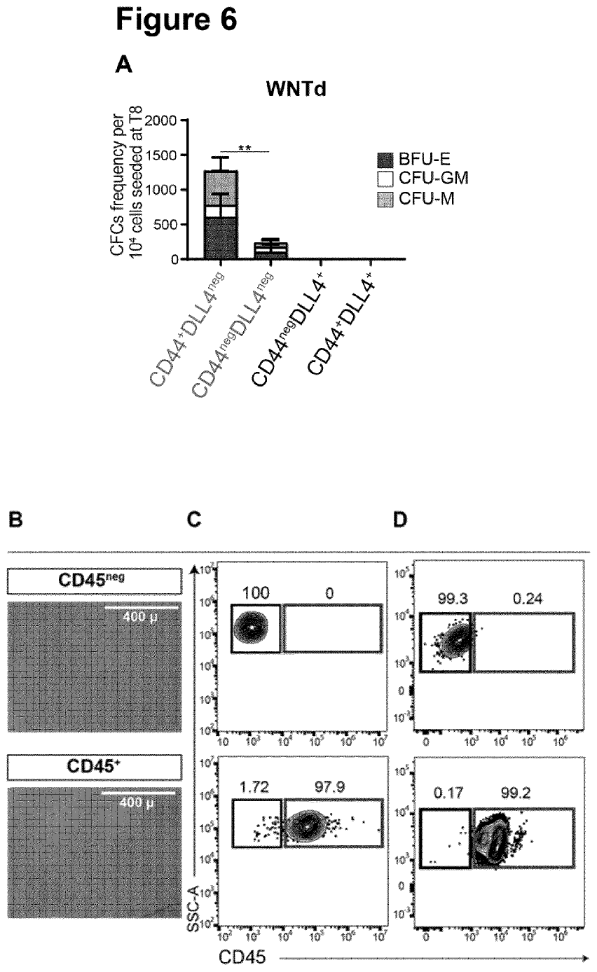

A) Experimental layout: CD34⁺CD43^{neg}CD45^{neg}CD32^{+/neg} (referred to as CD32+ and CD32^{neg}) cells were FAC-sorted from the AGM or YS of two CS13 human embryos. Isolated cells were seeded to assay their NK and T lymphoid, erythroid and myeloid (CFC generation assay) potential; B) Representative flow cytometric analysis of CD32+(orange) and CD32^{neg} (blue) cell populations within AGM and YS of CS13 human embryos. Gated on SSC/FSC/Live/CD34⁺CD43^{neg}CD45^{neg} as shown in FIG. 6A. n=2, independent; C) Quantification of erythro-myeloid CFC potential from CD32⁺ and CD32^{neg} populations isolated from the YS of two CS13 human embryos (E5, E6), as reported in E, F, 6A. D) Quantification of erythro-myeloid CFC potential from CD32⁺ and CD32^{neg} populations isolated from the AGM of two CS13 human embryos (E5, E6), as reported in E, F, 6A. BFU-E: burst forming unit erythroid; CFU-GM: colony forming unit granulocyte macrophage, CFU-M: colony forming unit macrophage; CFU-GEMM: colony forming unit granulocytes, erythrocytes, macrophages, megakaryocytes.

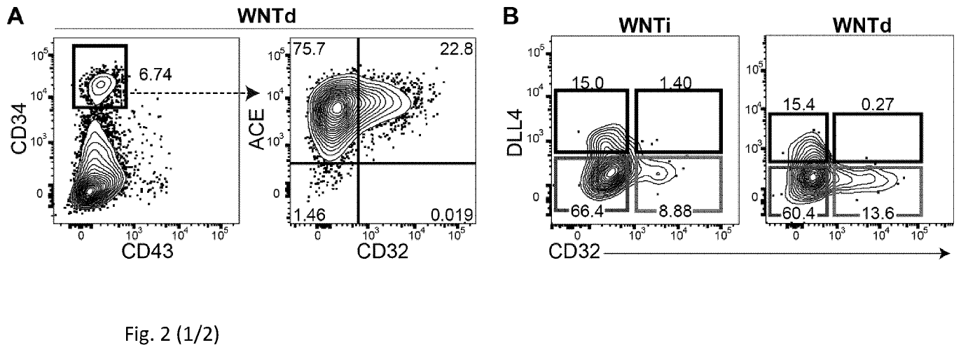

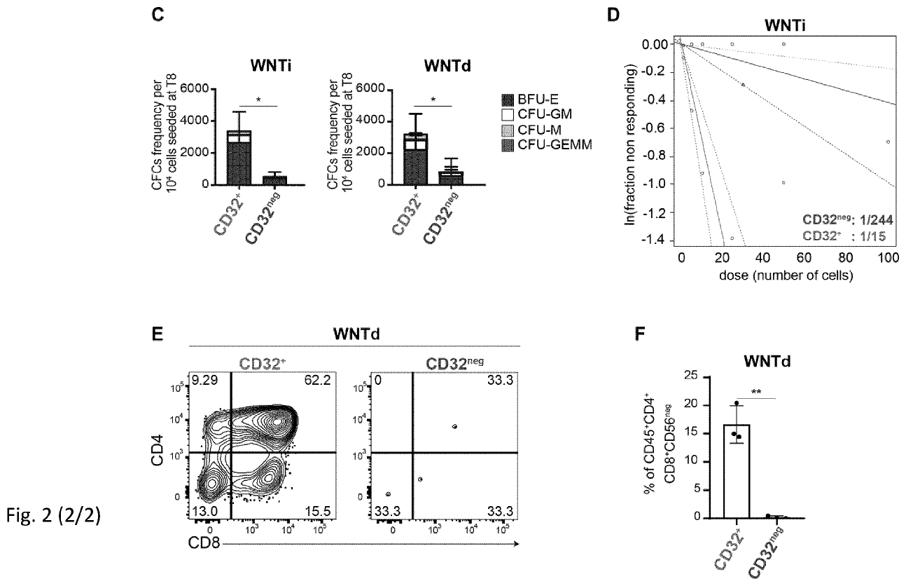

FIG. 2: CD32 is expressed in HECs of hPSC differentiation cultures.

A) Representative flow cytometric analysis of day 8 WNTd hPSC-derived hematopoietic cultures. Left panel: CD43 and CD34 expression gated on SSC/FSC/Live. Right panel: CD32 and ACE expression gated on SSC/FSC/Live/CD34⁺CD43^{neg} cells. n=3, independent; B) Representative flow cytometric analysis of CD32 and DLL4 expression within CD34⁺CD43^{neg}CD184^{neg}CD73^{neg} cells at day 8 of WNTi (left panel) and WNTd (right panel) hPSC-derived hematopoietic cultures. Gated on SSC/FSC/Live/CD34⁺CD43^{neg}CD184^{neg}CD73^{neg}. Four different populations are highlighted: CD32⁺DLL4^{neg} (referred to as CD32⁺) in orange, CD32^{neg}DLL4^{neg} (referred to as CD32^{neg}) in blue, CD32^{neg}DLL4⁺ and CD32⁺DLL4⁺ in black. n=7, independent; C) Quantification of erythro-myeloid CFC potential of CD32⁺ and CD32^{neg} populations isolated from day 8 WNTi (left panel) or WNTd (right panel) hPSC-derived hematopoietic cultures. One-tail paired Student's t-test, nonparametric, for all biological replicates (WNTi: n=6, WNTd, n=4), considering the total number of colonies, mean±SD, (WNTi: CD32⁺ vs CD32^{neg}, p=0.0111; WNTd: CD32^{neg} vs CD32⁺, p=0.0155). BFU-E: burst forming unit erythroid; CFU-GM: colony forming unit granulocyte macrophage, CFU-M: colony forming unit macrophage; CFU-GEMM: colony forming unit granulocytes, erythrocytes, macrophages, megakaryocytes; D) Frequency of NK-cell progenitors within the CD32⁺ (orange) and CD32^{neg} (blue) cell fractions isolated at day 8 of WNTi hPSC-derived hematopoietic cultures, from n=3 biological replicates; E) Representative flow cytometric analysis of CD4⁺CD8⁺ T-cell potential of CD32⁺ (orange) and CD32^{neg} (blue) cells isolated at day 8 of WNTd hPSC-derived hematopoietic cultures. Gated on SSC/FSC/Live/CD45⁺CD56^{neg}. n=3, independent; F) Bar plot showing the frequency of CD45⁺CD4⁺CD8⁺CD56^{neg} T-cells ratioed on the frequency of CD45⁺CD56^{neg} cells from CD32 factions. One-tail paired Student's t-test, nonparametric, for all biological replicates (n=3), mean±SD, **p<0.01.

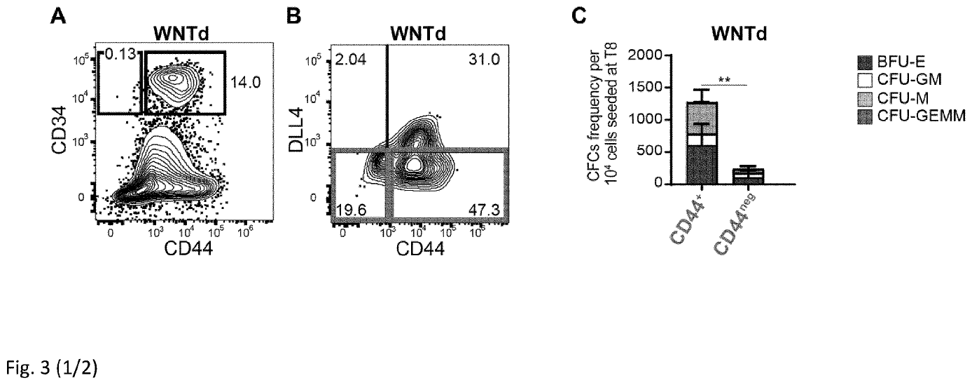

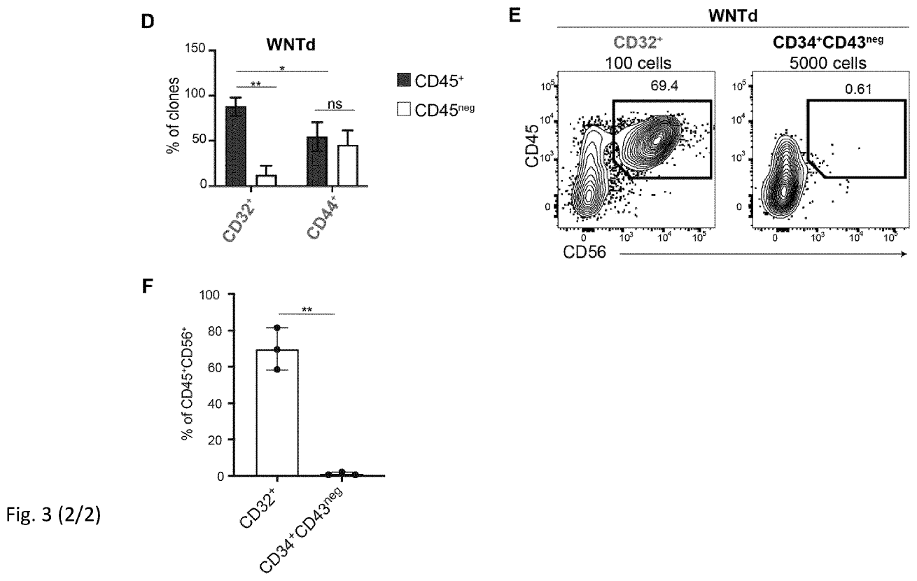

FIG. 3: CD32 is a highly specific marker for hPSC-derived HECs.

A) Representative flow cytometric analysis of CD44 and CD34 expression in day 8 WNTd hPSC-derived hematopoietic cultures. Gated on SSC/FSC/Live. n=3, independent; B) Representative flow cytometric analysis of CD44 and DLL4 expression within CD34⁺CD43^{neg}CD73^{neg}CD184^{neg} cells at day 8 of WNTd hPSC.-derived hematopoietic cultures. Two populations are highlighted within DLL4^{neg} fraction: CD44⁺ in green and CD44^{neg} in purple. Gated on SSC/FSC/Live/CD34⁺CD43^{neg}CD184^{neg}CD73^{neg}. n=3, independent; C) Quantification of erythro-myeloid CFC potential of CD44⁺ (green) and CD44^{neg} (purple) populations isolated at day 8 of WNTd hPSC-derived hematopoietic cultures. One-tail paired Student's t-test, nonparametric, for all biological replicates (n=3) considering the total number of colonies, mean±SD, (CD44⁺ vs CD44^{neg}, p=0.0098). BFU-E: burst forming unit erythroid; CFU-GM: colony forming unit granulocyte macrophage, CFU-M: colony forming unit macrophage; CFU-GEMM: colony forming unit granulocytes, erythrocytes, macrophages, megakaryocytes; D) Bar plot showing the frequency of CD45⁺ (magenta) and CD45^{neg} (white) clones derived from CD44+(green) or CD32⁺ (orange) cells isolated at day 8 of WNTd hematopoietic cultures. One-tail paired Student's t-test for all biological replicates (n=3), mean±SD, (for CD44⁺: CD45⁺ vs CD45^{neg}, p=0.3393; for CD32⁺: CD45⁺ vs CD45^{neg}, p=0.0068905). One-tail unpaired Student's t-test for CD44⁺ vs CD32⁺ p=0.014553. E) Representative flow cytometric analysis of CD45⁺CD56⁺ NK-cells derived from corrected SCID-X1 iPSCs when either 5000 CD34⁺CD43^{neg}(left panel) or 100 CD34⁺CD43^{neg}CD184^{neg}CD73^{neg}DLL4^{neg}CD32⁺ cells are seeded at day 8 of WNTd hPSC-derived hematopoietic cultures; n=3, independent. F) Bar plot showing the frequency of CD45⁺CD56⁺ NK-cells derived from CD34⁺CD43^{neg}CD184^{neg}CD73^{neg}DLL4^{neg}CD32⁺ (referred to as CD32⁺) and CD34⁺CD43^{neg} fractions. One-tail paired Student's t-test, nonparametric, for all biological replicates (n=3), mean±SD,**p<0.01.

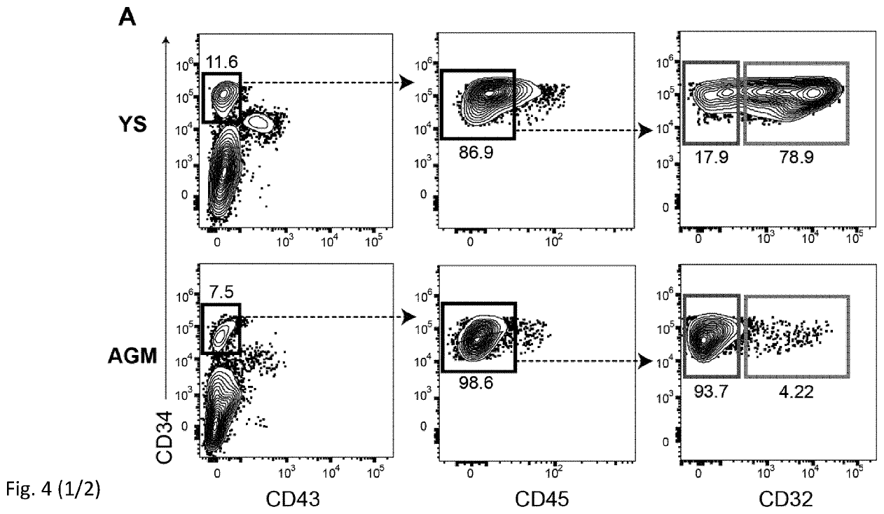

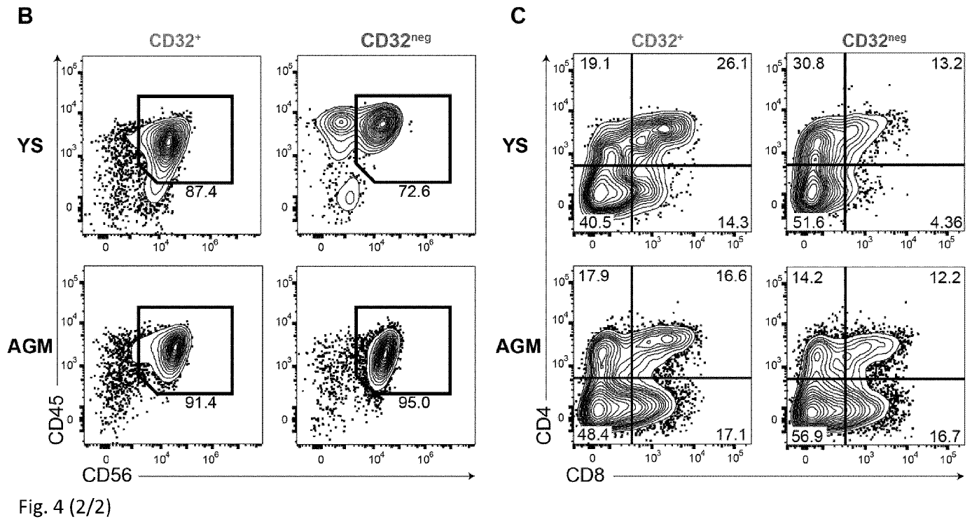

FIG. 4

A) Representative flow cytometric analysis showing the gating strategy to isolate CD32⁺ (orange) and CD32^{neg} (blue) cells from the YS (upper panels) or the AGM (bottom panel) of CS13 human embryo. Left panel: gated on SSC/FSC/Live. Middle panel: gated in SSC/FSC/Live/CD34⁺CD43^{neg}. Right panel: gated in SSC/FSC/Live/CD34⁺CD43^{neg}CD45^{neg}. n=2, independent; B) Representative flow cytometric analysis of the CD45⁺CD56⁺ NK-cell potential of CD32⁺ (orange) and CD32^{neg} (blue) cells isolated from the YS (upper panels) or AGM (bottom panels) of CS13 human embryos. Gated on SSC/FSC/Live. n=2, independent; C) Representative analysis of the CD4⁺CD8⁺ T-cell potential of CD32⁺ (orange, left column) and CD32^{neg} (blue, right column) cells isolated from the YS (upper panels) or AGM (bottom panels) of CS13 human embryos. Gated on SSC/FSC/Live/CD45⁺CD56^{neg}. n=2, independent.

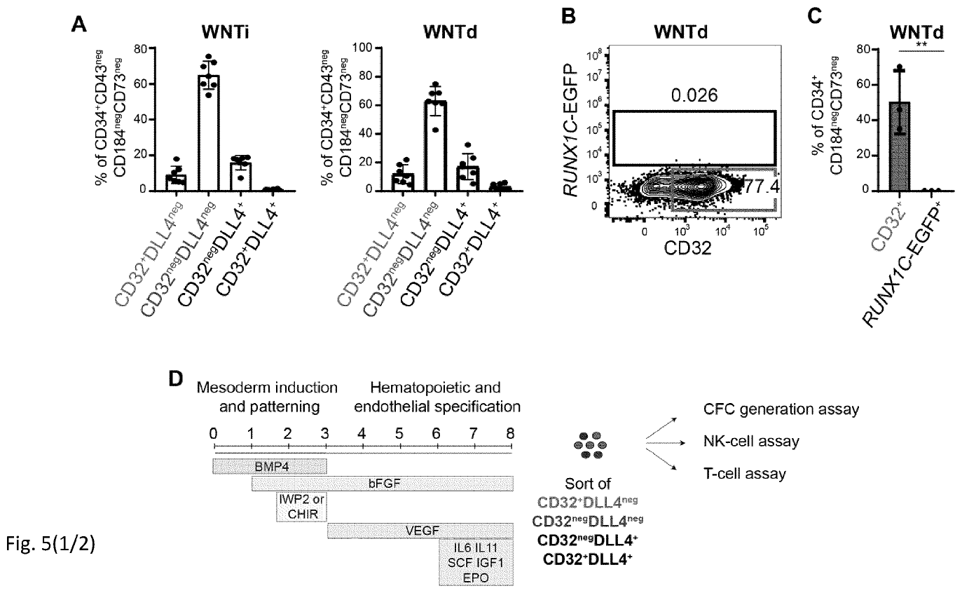

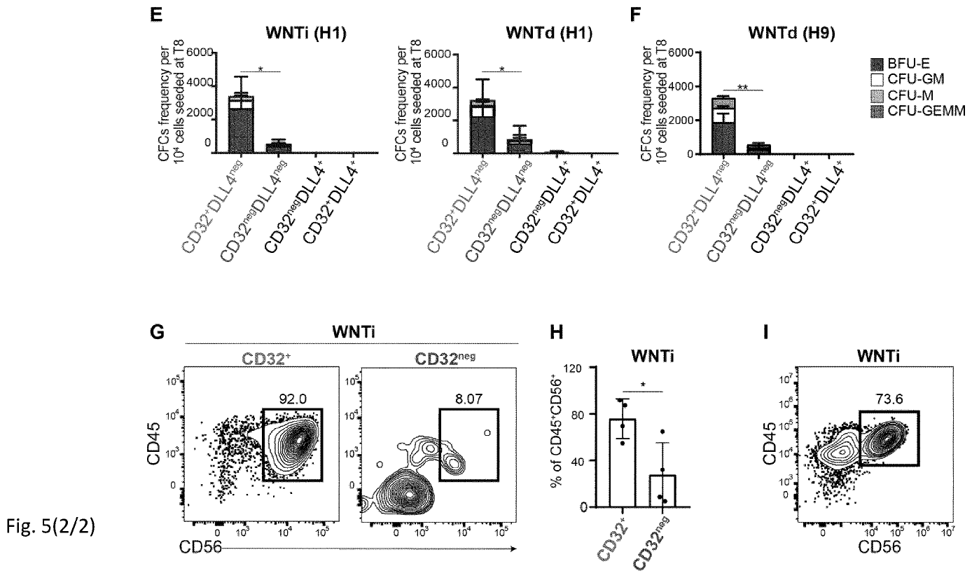

FIG. 5

A) Bar plot showing the frequency of CD32^{+/neg}DLL4^{+/neg} cells within day 8 of WNT-independent (left panel) or WNT-dependent (right panel) hPSC-derived CD34⁺CD43^{neg}CD184^{neg}CD73^{neg} cells. Gated on SSC/FSC/Live/CD34⁺CD43^{neg}CD184^{neg}CD73^{neg}. n=7, independent; B) Representative flow cytometric analysis showing CD32 and RUNX1C-EGFP expression in day 8 WNTd hPSC-derived CD34⁺CD184^{neg}CD73^{neg} cells. Gated on SSC/FSC/Live/CD34⁺CD184^{neg}CD73^{neg}. n=3, independent; C) Bar plot showing CD32 and RUNX1C-EGFP expression in day 8 WNTd CD34⁺CD184^{neg}CD73^{neg} cells in n=3 experiments, as shown in B. Gated on SSC/FSC/Live/CD34⁺CD184^{neg}CD73^{neg}; D) Experimental layout showing the timeline of WNTi or WNTd hPSC-derived hematopoietic cultures obtained by adding a WNT antagonist (IWP2) or WNT agonist (CHIR 99021) respectively. CD34⁺CD43^{neg}CD184^{neg}CD73^{neg}CD32^{+/neg}DLL4^{+/neg} (referred to as CD32^{+/neg}DLL4^{+/neg}) cells were isolated at day 8 and further cultured to assay the CFC generation, NK- or T-lymphoid potential; E) Quantification of erythro-myeloid CFC potential of CD32^{+/neg}DLL4^{+/neg} populations isolated at day 8 of WNTi (left panel) or WNTd (right panel) hPSC-derived hematopoietic cultures performed on H1 hESC line. Statistical analysis performed as in FIG. 3C (WNTi, n=6; WNTd, n=4) as the DLL4^{neg} have a null value. F) Quantification of erythro-myeloid CFC potential of CD32^{+/neg}DLL4^{+/neg} populations isolated at day 8 of WNTd hPSC-derived hematopoietic cultures performed on H9 hESC line. One-tail paired Student's t-test for all biological replicates (n=3) considering the total number of colonies, mean±SD, (H9 CD32⁺DLL4^{neg} vs CD32^{neg} DLL4^{neg}, p=0.0092). DLL4^{neg} have not been considered as they have a null value. BFU-E: burst forming unit erythroid; CFU-GM: colony forming unit granulocyte macrophage, CFU-M: colony forming unit macrophage; CFU-GEMM: colony forming unit granulocytes, erythrocytes, macrophages, megakaryocytes; G) Representative flow cytometric analysis showing the CD45⁺CD56⁺ NK-cell potential of CD32⁺ (orange) and CD32^{neg} (blue) fractions isolated at day 8 of WNTi hPSC-derived hematopoietic cultures. Gated on SSC/FSC/Live. n=3, independent; H) Bar plot showing the frequency of CD45⁺CD56⁺ NK-cells from of CD32⁺ (orange) and CD32^{neg} (blue) fractions as shown in G. One-tail paired Student's t-test, nonparametric, for all biological replicates (n=4), mean±SD, *p<0.05. I) Representative flow cytometric analysis showing CD45⁺CD56⁺ NK-cells derived from a single CD32⁺ cell isolated at day 8 of WNTi hematopoietic. Gated on SSC/FSC/Live. n=3, independent.

FIG. 6

A) Quantification of erythro-myeloid CFC potential of CD34⁺CD43^{neg}CD184^{neg}CD73^{neg}CD44^{+/neg}DLL4^{+/neg} cells isolated at day 8 of WNTd hPSC-derived hematopoietic cultures. Statistical analysis performed as in FIG. 4C (n=3) as the DLL4^{neg} have a null value. BFU-E: burst forming unit erythroid; CFU-GM: colony forming unit granulocyte macrophage, CFU-M: colony forming unit macrophage; CFU-GEMM: colony forming unit granulocytes, erythrocytes, macrophages, megakaryocytes; B) Photo-micrograph of a clone composed by adherent non-hematopoietic (upper panel) or round hematopoietic cells (bottom panel) derived from single CD34⁺CD43^{neg}CD184^{neg}CD73^{neg}CD32⁺ cells isolated at day 8 of WNTd hPSC-derived hematopoietic culture; C) Representative flow cytometric analysis of a CD45^{neg} (upper panel) or CD45⁺ (bottom panel) clone derived from single CD34⁺CD43^{neg}CD184^{neg}CD73^{neg}CD32⁺ cell isolated at day 8 of WNTd hPSC-derived hematopoietic culture. Gated on SSC/FSC/Live. n=3, independent; D) Representative flow cytometric analysis of a CD45^{neg} (upper panel) or CD45⁺ (bottom panel) clone derived from single CD34⁺CD43^{neg}CD184^{neg}CD73^{neg}CD44⁺ cell isolated at day 8 of WNTd hPSC-derived hematopoietic culture. Gated on SSC/FSC/Live. n=3, independent.

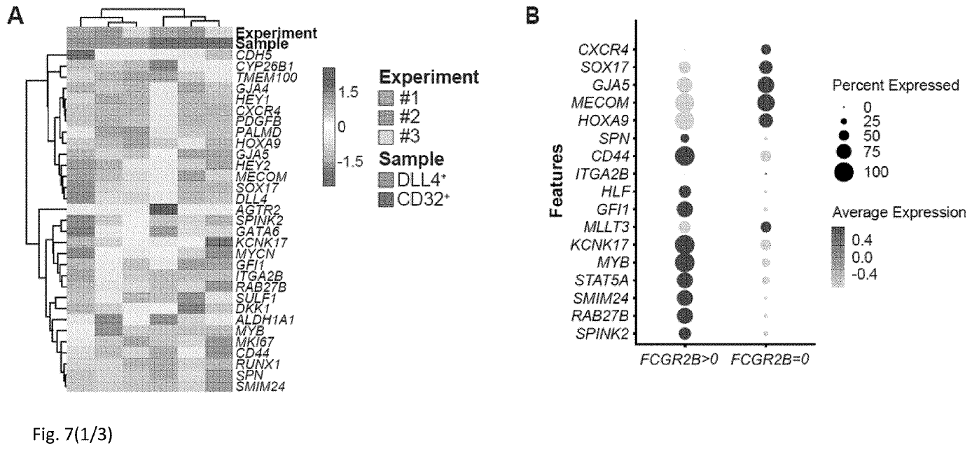

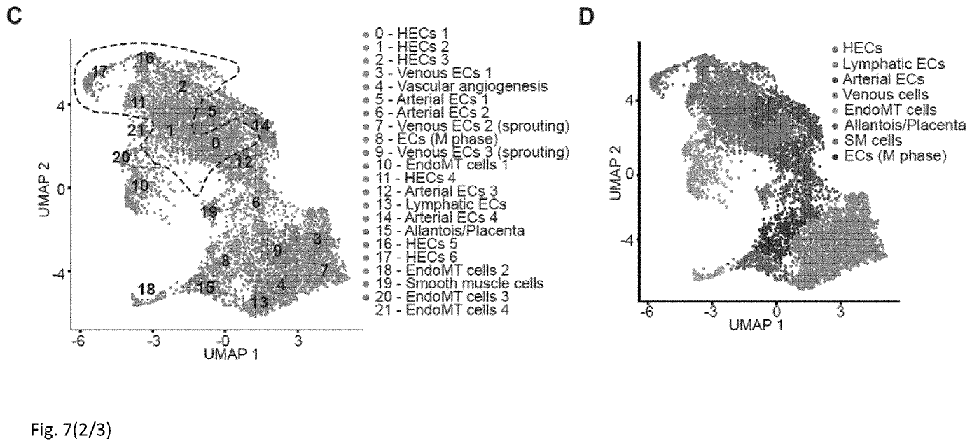

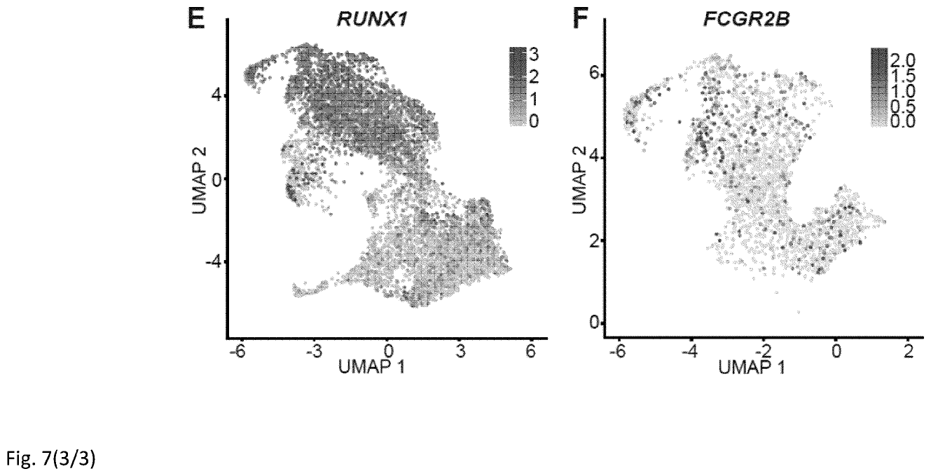

FIG. 7

A) Heatmap showing a selection of differentially expressed genes in CD34⁺CD184⁺CD73⁺DLL4⁺CD43^{neg} (referred to as DLL4⁺, in green) or CD34⁺CD43^{neg}CD184^{neg}CD73^{neg}CD32⁺ (referred to as CD32⁺, in orange) samples isolated at day 8 of WNTd hPSC-derived hematopoietic culture in n=3 independent experiments (#1 in light blue, #2 in purple, #3 in yellow) performed in H1 hESCs. Scaled rlog gene expression values are shown in rows. Tiles referring to differentially expressed genes are coloured according to up- (red) or down- (blue) regulation; B) Scorecard dot plot showing landmark genes as reported in (Calvanese et al, 2022). The differential expression was evaluated in CDH5⁺RUNX1⁺PTPRC^{neg} cells that either express FCGR2B (FCGR2B>0) or not (FCGR2B=0); C) UMAP of single-cell data of CD34⁺CD43^{neg}CD184^{neg}CD73^{neg} cells isolated at day 8 WNTd hPSC-derived hematopoietic cultures, H1 hESCs, n=1. Cells are clustered at resolution 0.6. RUNX1 expressing clusters are highlighted by a black dashed line. HECs: hemogenic endothelial cells; ECs: endothelial cells; EndoMT: endothelial-to-mesenchyme transition; M phase: mitotic phase; D) UMAP visualization of manually annotated cells and colour-coded by cell type. H1 hESCs, n=1. HECs: hemogenic endothelial cells; ECs: endothelial cells; EndoMT: endothelial-to-mesenchyme transition; M phase: mitotic phase; E) Feature plot showing RUNX1 expression across clusters of single-cell data set in FIG. 7a; F) Feature plot showing FCGR2B expression across clusters 0, 1, 2, 11, 16, 17 that display RUNX1 as a differential marker;

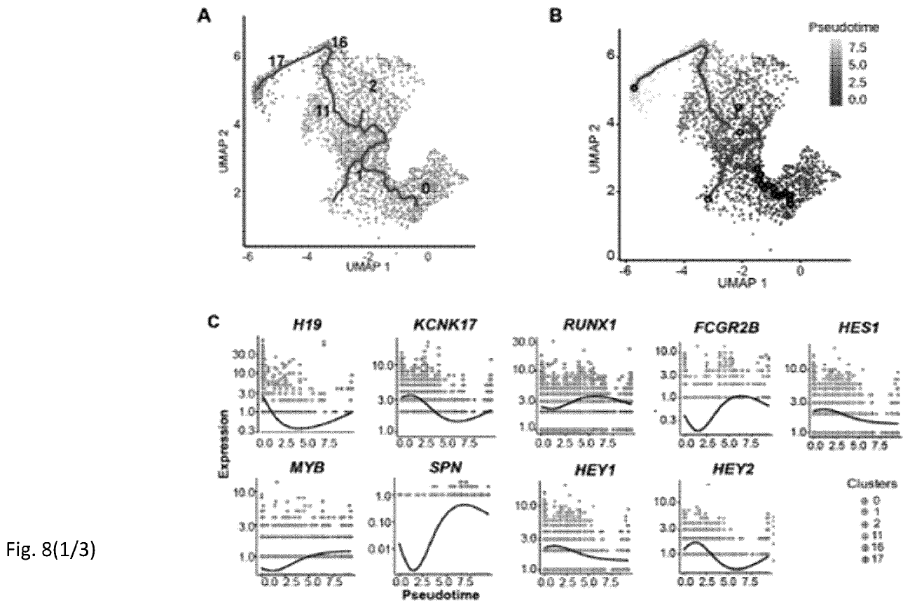

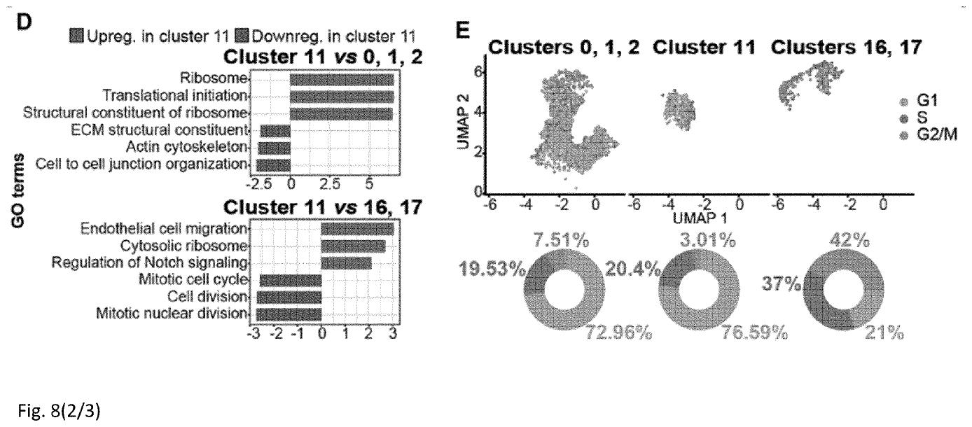

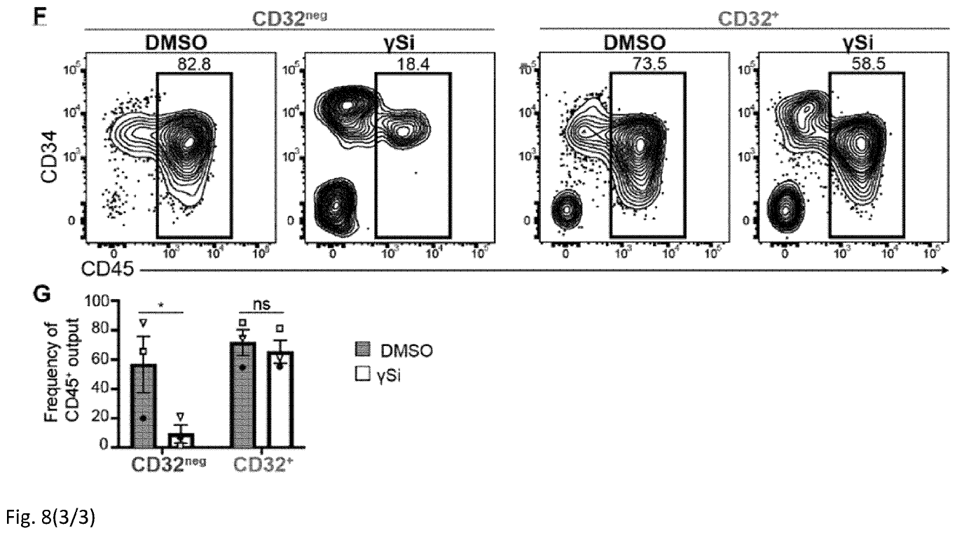

FIG. 8.

A) UMAP visualization of the single-cell trajectory performed by Monocle3 on clusters 0, 1, 2, 11, 16, 17 that display RUNX1 as a differential marker; B) UMAP visualization of the pseudotime analysis by monocle3 showing the principal graph nodes and trajectory on the clusters with differential expression of RUNX1 as obtained by scRNA sequencing. Cells are coloured according to pseudotime; C) Pseudotime kinetics of the expression variation of selected genes (H19, KCNK17, RUNX1, MYB, SPN, FCGR2B and the Notch targets genes HES1, HEY1 and HEY2) along the clusters with differential expression of RUNX1 (clusters 0, 1, 2, 11, 16, 17). Cells are coloured by the cluster identity. Lines denote relative average expression of each gene in pseudotime; D) Barplot showing significantly enriched Gene Ontology (GO) terms using GSEA (adjusted p-value<0.05) on fold change pre-ranked genes from the comparison between cluster 11 and clusters 0, 1, 2 (upper panel) or cluster 11 and clusters 16, 17 (bottom panel). Upregulated (upreg.) genes are shown in red, downregulated (downreg.) in blue. NES: normalized enrichment score, ECM: extracellular matrix: E) Upper panel: UMAP showing the cells in clusters 0, 1, 2, cluster 11, and cluster 16, 17 coloured according to the cell cycle phase. G1 phase is shown in yellow, S in purple, G2/M in green. Lower panel: donut charts showing the percentage of cells in G1, G2/M, and S phase in each of the clusters; F) Representative flow cytometric analysis of the hematopoietic CD45⁺ and endothelial CD34⁺ progeny derived after 4 days of HEC culture of CD32^{+/neg} cells isolated at day 8 of WNTd hPSC-derived hematopoietic cultures and treated with DMSO, as control, or γ-secretase inhibitor L-685,458 (γSi) to block Notch signaling. Gated on SSC/FSC/Live. n=3, independent; G) Bar plot showing the frequency of CD45⁺ cells derived from CD32⁺ and CD32^{neg} fraction treated with DMSO (in petroleum) or γ-secretase inhibitor L-685,458 (γSi, in white) to block Notch signaling. One-way ANOVA for all biological replicates (n=3), mean±SEM (*p=0.0456; ns, not significant, p=0.9623).

### DETAILED DESCRIPTION OF THE INVENTION

The present invention provides a reservoir of hemogenic endothelial cells (HEC) that can give rise to hematopoietic cells, and surface markers that allows separation of HECs from other cell types. HECs are found in the endothelial cell layers of several organs and have the ability to reconstitute the immune system for the treatment of hematopoietic disorders.

As described herein, the in vitro generation of hematopoietic progenitors can provide either patient-specific cell-based therapeutics, or “off-the-shelf” universal donor products. The disclosed methodology to produce in vitro derived HCs can be easily implemented, is robust, and can be used in the development of various clinical and industrial applications, such as but not limited to: cell-based therapies for a variety of hematological conditions; scalable generation of lymphoid progenitors and terminally differentiated lymphocytes for adoptive immunotherapy; scalable generation of megakaryocyte progenitors and/or platelets for transfusion; scalable generation of erythroid progenitors and/or mature erythrocytes for transfusion; the generation of HSCs as a substitute for bone marrow transplantation; drug/toxicity screening on any progenitor or terminally differentiated hematopoietic cell; gene therapy or gene-correction and allogeneic transplant of patient-derived hPSCs. The present cell-based therapeutics include CAR T, CAR NK, NK and regulatory T cells.

The invention provides also a method that may comprise the following steps:
(i) differentiating a population of pluripotent stem cells (PSCs), preferably into mesoderm cells, and;
(ii) Isolating CD32+ cells from the population of cells obtained from step (i) to obtain hemogenic endothelial cells (HECs) or enriched populations of HECs.

The PSCs are preferably differentiated, preferably into mesoderm cells, by culturing the population of PSCs under suitable conditions to promote and/or induce mesodermal differentiation.

The mesoderm cells may be differentiated into HECs by culturing the population of mesoderm cells under suitable conditions to promote and/or induce hemogenic endothelial (HE) differentiation.

Therefore, the method of the invention may further comprise a step of differentiating the mesoderm cells into HECs by culturing the population of mesoderm cells under suitable conditions to promote and/or induce hemogenic endothelial (HE) differentiation.

The invention therefore also provides a method for obtaining hemogenic endothelial cells (HECs) or enriched populations of HECs comprising the steps of:
i. Differentiating a population of pluripotent stem cells (PSCs) into mesoderm cells, preferably by culturing population of pluripotent stem cells in a mesoderm differentiation medium and/or hematovascular differentiation medium to obtain a population of mesoderm cells;
ii. Differentiating the mesoderm cells obtained in step i. into HECs, preferably by culturing the population of mesoderm cells under suitable conditions to promote and/or induce hemogenic endothelial (HE) differentiation,
iii. Isolating CD32+ cells from the population of cells obtained from step (ii) to obtain hemogenic endothelial cells (HECs) or enriched populations of HECs.

The HECs may be differentiated into HPCs or HCs by culturing the population of HECs under suitable conditions to promote hematopoietic differentiation.

Preferably the HECs are cultured in a hematopoietic induction medium to promote and/or induce differentiation into HPCs or HCs.

The method of the invention may further comprises differentiating the population of HPCs into progenitor NK or T cells and optionally comprises maturing the progenitor NK or T cells to produce a population of NK or T cells, respectively.

The method of the invention may further comprise introducing heterologous nucleic acid encoding an antigen receptor into the PSCs, iPSCs, HPCs or progenitor T cells.

Preferably the heterologous nucleic acid encoding the antigen receptor, said nucleic acid including DNA or mRNA, is comprised in an expression vector, more preferably it is a lentiviral vector or adeno-associated viral (AAV) vector.

Preferably the heterologous nucleic acid is incorporated into the genome of the PSCs, iPSCs, HECs, hematopoietic progenitor cells, or progenitor T cells using a gene editing system, e.g. CRISPR/Cas9 or AAV.

Preferably the antigen receptor is a TCR, preferably the TCR is an affinity enhanced TCR or a non-MHC restricted TCR.

Preferably the antigen receptor is a chimeric antigen receptor (CAR) or NKCR.

Preferably the CAR or NKCR binds specifically to a target antigen expressed by cells, preferably it binds specifically to an MHC displaying a peptide fragment of a tumour antigen expressed by cancer cells.

The expression “the cells or the population is CD32+CD34+CD43−CD184−CD73−DLL4−” means e.g. that the cell or the population may be positive for CD32 and/or CD34 and/or be negative for CD43 and/or CD184 and/or CD73 and/or DLL4, preferably the cell or the population may be positive for CD32 and CD34 and may be negative for CD43 and CD184 and CD73 and DLL4. “Hemogenic endothelial cells” (HECs) are endothelial cells that have the capacity to generate hematopoietic cells, including hematopoietic stem cells.

HECs disclosed herein can be defined by expression of the marker CD32 (cluster of differentiation 33). CD32⁺ expression defines HECs.

HECs can further optionally be defined by expression, in addition to CD32, of one or more additional markers selected from CD34, CD31, CD54, CD102, CD47, CD144, CD146, CD124, CD201, CD151, LTBR, CD39, CD147, CD105, CD143, CD309, CD44.

Preferably, the cells show positive expression of one or more of these additional markers. HECs can be defined by expression of 1, 2, 3, 4, 5, 6, 7, 8, or all of these additional markers, in addition to CD32+ expression.

Preferably HECs do not express CD43, CD184, CD73 and/or DLL4.

HECs have some characteristics of endothelial cells (ECs), for example, expression of the EC marker CD 144 and optionally, expression of the EC marker CD31 (also known as platelet endothelial cell adhesion molecule-1 or PECAM-1).

The marker genes and the sequences of those genes and the corresponding markers, which are incorporated by reference herein, are found in the publicly available GenBank database by virtue of their gene identification numbers or Entrez Gene ID designations or NCBI Reference Sequence Accession number. Accordingly, all GenBank gene identification numbers and sequences related thereto are incorporated by reference in their entirety herein.

In the context of the present invention the “marker” or the protein herein mentioned refer to, without limitation, the mentioned proteins (and sequence information thereof or above mentioned), and to peptides, nucleic acids, oligonucleotides thereof together with their related metabolites, mutations, variants, orthologues, polymorphisms, modifications, fragments, subunits, isoforms, precursors, degradation products, elements, and other analytes or sample-derived measures. Markers can also include mutated proteins, mutated nucleic acids, variations in copy numbers and/or transcript variants. The term “does not express” also includes the term expresses low/no level.

Cell comprising a particular cell surface marker can be isolated by Fluorescence activated Cell Sorting (FACS) purification and/or using a marker specific affinity reagent. For example, the affinity reagent may be conjugated to beads. In some embodiments, the methods comprise one or multiple purification rounds for example one or more multiple FACS purifications or affinity beads based for example on cell surface expression patterns described herein.

In one embodiment, the beads are magnetic beads for magnetic based separation of cells, optionally polystyrene spherical beads that are superparamagnetic.

In some embodiments, one or more enrichment steps are performed.

In one embodiment, the population is an isolated population and/or an in vitro produced population. For example, an aspect includes an in vitro produced cell according to a method described herein.

The expression of cell markers may be determined by any suitable technique, including immunocytochemistry, immunofluorescence, RT-PCR, immunoblotting, fluorescence activated cell sorting (FACS), and enzymatic analysis. In preferred embodiments, a cell may be said to express a marker if the marker is detectable on the cell surface. For example, a cell which is stated herein not to express a marker may display active transcription and intracellular expression of the marker gene but detectable levels of the marker may not be present on the surface of the cell.

As described herein, the methods produced cell populations that are enriched for a particular cell type. In an embodiment, the methods involve producing a population comprises at least 30%, at least 35%, at least 40% or at least 50%, of the desired cell type. In other embodiments cells can be isolated to provide for example greater numbers, for example at least 60%, at least 70%, at least 80% or at least 90% of cells expressing one or more markers described herein. As used herein, the term “enrich” is meant to refer to increasing the proportion of a desired substance, for example, to increase the relative frequency of a subtype of cell or cell component compared to its natural frequency in a cell population. Positive selection, negative selection, or both are generally considered necessary to any enrichment scheme. Selection methods include, without limitation, magnetic separation and fluorescence-activated cell sorting (FACS).

In the present invention, “expressing” (or cell positive for a specific marker) means that the expression can be detected by the method used for separating or isolating cells, and “not expressing” (or cell negative for a specific marker) means that the detection is not possible with the detection sensitivity of the method used for separating or isolating cells.

“Hematopoietic cells” (HCs) are the cells that can be generated from HECs, including hematopoietic stem cells (HSCs).

The hematopoietic stem cell (HSC) is multipotent and ultimately gives rise to all types of terminally differentiated blood cells. The hematopoietic stem cell can self-renew, or it can differentiate into more committed progenitor cells, which progenitor cells are irreversibly determined to be ancestors of only a few types of blood cell. For instance, the hematopoietic stem cell can differentiate into (i) myeloid progenitor cells, which myeloid progenitor cells ultimately give rise to monocytes and macrophages, neutrophils, basophils, eosinophils, erythrocytes, megakaryocytes/platelets, dendritic cells, or (ii) lymphoid progenitor cells, which lymphoid progenitor cells ultimately give rise to T-cells, B-cells, and lymphocyte-like cells called natural killer cells (NK-cells). Once the stem cell differentiates into a myeloid progenitor cell, its progeny cannot give rise to cells of the lymphoid lineage, and, similarly, lymphoid progenitor cells cannot give rise to cells of the myeloid lineage. For a general discussion of hematopoiesis and hematopoietic stem cell differentiation, see Chapter 17, Differentiated Cells and the Maintenance of Tissues, Alberts et al., 1989, Molecular Biology of the Cell, 2^{nd} Ed., Garland Publishing, New York, N.Y.; Chapter 2 of Regenerative Medicine, Department of Health and Human Services, August 2006, and Chapter 5 of Hematopoietic Stem Cells, 2009, Stem Cell Information, Department of Health and Human Services.

In vitro and in vivo assays have been developed to characterize hematopoietic stem and progenitor cells, for example, the spleen colony forming (CFU-S) assay and reconstitution assays in immune-deficient mice. Further, presence or absence of cell surface protein markers defined by monoclonal antibody recognition have been used to recognize and isolate hematopoietic stem cells. Such markers include, but are not limited to, Lin, CD34, CD38, CD43, CD45RO, CD45RA, CD59, CD90, CD109, CD117, CD133, CD166, and HLA DR, and combinations thereof. See Chapter 2 of Regenerative Medicine, Department of Health and Human Services, August 2006, and the references cited therein.

In the context of the present invention the term “hematopoietic cells” comprises “hematopoietic progenitor cells” and “hematopoietic stem and progenitor cells (HSPCs)”. In some embodiments such terms may be used interchangeably.

In the context of the present invention the term “hematopoietic cells” may also comprise “blood progenitor cells” and “hematopoietic precursors”. In some embodiments such terms may be used interchangeably.

“Hematopoietic cells” encompass myeloid lineage cells, which include erythrocytes, monocytes, macrophages, megakaryocytes, myeloblasts, dendritic cells, and granulocytes (basophils, neutrophils, eosinophils, and mast cells); and lymphoid lineage cells, which include T lymphocytes/T cells, B lymphocytes/B cells, and natural killer cells. The HECs disclosed herein have the ability to generate HCs including myeloid lineage cells and lymphoid lineage cells.

In the present invention the isolation step may be preceded by a step wherein cells positive for the marker CD32 (and optionally other markers herein mentioned) are identified. Identification may be performed for example, using antibodies or antigen-binding fragments thereof that bind to cell surface markers, conjugated to an imaging moiety such as a fluorescent or magnetic agent, a radioisotope, or other suitable imaging agent.

In the context of the present invention the term “a population of cells derived from pluripotent stem cells” may be a population of pluripotent stem cells undergoing differentiation or which is treated and/or cultured to differentiate towards hematopoietic cells or HECs. Such term may include cultured cells before or after the differentiation. The term may also include a population of pluripotent stem cell undergoing differentiation in mesodermal cells which are treated in a cytokine-defined culture to further differentiate towards hematopoietic stem cell (HSC).

The terms “isolated” and “purified” are used interchangeably herein to refer to a material that is substantially or essentially removed from or concentrated from its natural environment. For example, a cell is isolated if it is substantially removed from other endogenous cell types, tissues, and materials which the cell would normally be found in proximity to in a subject. Methods for purification and isolation of cell types according to expression of cell-surface markers are documented methodologies. A “substantially isolated” cell or cell population is a cell or cell population that is at least 50%, 60%, 70%, 80%, 85%, 90%, 95%, 98%, or 99% or more isolated from other cell types, tissues, or materials found in the tissue of a subject. Also, a cell or cell population is “substantially purified” when at least 50%, 60%, 70%, 80%, 85%, 90%, 95%, 98%, or 99% or more of the cells in a cell sample express the cell-surface markers of interest.

In an exemplary method, as a first step, HECs are isolated from hPSC cultures according to CD32+ expression, optionally in combination with one or more additional markers as disclosed above. Methods to isolate or separate cells according to expression of cell surface markers include: fluorescence activated cell sorting by the use of e.g. antibodies or fragments thereof directed to CD32, and optionally using additional antibodies or fragments thereof directed to additional markers; magnetic separation, using e.g. antibody-coated magnetic beads; affinity chromatography using antibodies or fragments thereof; “panning” with antibodies or fragments thereof attached to a solid matrix, e.g., a plate or other solid matrix; or other techniques as are known and used in the art for separation of cells based on cell surface marker expression. In preferred embodiments, fluorescence activated cell sorting or magnetic separation is used to isolate HECs from non-HECs.

Isolation of HECs generates a substantially pure population of CD32+ cells. As defined herein, a “substantially pure population” of CD32+ cells means e.g. that more than 50%, more than 60%, more than 70%, more than 75%, more than 80%, more than 85%, more than 90%, more than 95%, more than 98%, more than 99%, or even 100% of the cells following the isolation/separation step are CD32+. As defined herein, an “enriched populations of HECs” means e.g. that more than 50%, more than 60%, more than 70%, more than 75%, more than 80%, more than 85%, more than 90%, more than 95%, more than 98%, more than 99%, or even 100% of the cells are HECs and/or are CD32+.

As defined herein, an “enriched populations” means e.g. that more than 50%, more than 60%, more than 70%, more than 75%, more than 80%, more than 85%, more than 90%, more than 95%, more than 98%, more than 99%, or even 100% of the cells are the particular desired cells. Following isolation, HECs can be maintained in culture for up to one week in Stempro media (Life Technologies, 10639011), complemented with 1% glutamine, 50 μg/ml ascorbic acid, 150 μg/ml transferrin, 400 μM 1-Thioglycerol solution, 30 ng/ml TPO, 5 ng/ml VEGF, 5 ng/ml bFGF, 30 ng/ml IL3, 100 ng/ml SCF, 25 ng/ml IGF1, 10 ng/ml IL6, 5 ng/ml IL11, 2 U/ml EPO, 10 ng/ml BMP4. Aggregates can then be transferred onto thin-layer Matrigel-coated plasticware where they were cultured for an additional 3-10 days in the same media.

Isolated HECs can be cryopreserved using techniques known in the art for cell cryopreservation. HECs can be frozen for storage, either directly after isolation, or following maintenance in culture conditions as described above, or after proliferation in culture.

Accordingly, in one embodiment, HECs can be derived from PSCs and frozen, until such time as further use is determined, at which point HEC can be thawed and used to autologous or non-autologous transplantation in a subject in need of transplant, or differentiated into (or cultured to obtain) hematopoietic cells and administered as cell therapy to subjects in need thereof.

Isolated HECs can be prepared for cryogenic storage by addition of one or a combination of cryoprotective agents such as dimethyl sulfoxide (DMSO), glycerol, polyvinylpyrrolidine, polyethylene glycol, albumin, dextran, sucrose, ethylene glycol, i-erythritol, D-ribitol, D-mannitol, D-sorbitol, i-inositol, D-lactose, choline chloride, amino acids, methanol, acetamide, glycerol monoacetate, and inorganic salts. Addition of plasma or serum (e.g., to a concentration of 20-25%) may augment the protective effect of DMSO. HECs can be frozen, for example, in 60-40% growth media as disclosed above (e.g., RPMI 1650, MEM or DMEM) with 40-60% serum and 5-20% DMSO. In one embodiment, HECs are frozen in 50% growth media, 50% FCS (fetal calf serum) with 10% DMSO.

Preferably HECs are frozen with Bambanker or StemPro34 40%, 10% DMSO, 50% FCS.

It is another object of the present invention a substantially pure population of CD32+ hemogenic endothelial cells, wherein more than 50%, more than 60%, more than 70%, more than 75%, more than 80%, more than 85%, more than 90%, more than 91%, more than 92%, more than 93%, more than 94%, more than 95%, more than 96%, more than 97%, more than 98%, more than 99%, more than 99.5%, or even 100% of the cells following the isolation/separation step are CD32+.

Also contemplated are compositions that include a substantially pure population of CD32+ HECs. In one embodiment, the substantially pure population of CD32+ HECs is included in a composition with at least one cryoprotective agent, such as disclosed above. The cells in this composition can be in a frozen or unfrozen state. In another embodiment, the substantially pure population of CD32+ HECs is included in a composition with a suitable culture medium, such as the culture media disclosed for maintenance of HECs in vitro.

The composition of the invention may be a pharmaceutical composition.

Pharmaceutically acceptable carriers for practicing the invention include well known components such as, for example, culture media and phosphate buffered saline. Additional pharmaceutically acceptable carriers and their formulations are well-known and generally described in, for example, Remington's Pharmaceutical Science (18th Ed., ed. Gennaro, Mack Publishing Co., Easton, Pa., 1990) and the Handbook of Pharmaceutical Excipients (4th ed., Ed. Rowe et al. Pharmaceutical Press, Washington, D.C.), each of which is incorporated by reference.

Examples of compositions include liquid preparations for parenteral, subcutaneous, intradermal, intramuscular, or intravenous administration (e.g., injectable administration), such as sterile suspensions or emulsions. Such compositions may be in admixture with a suitable carrier, diluent, or excipient such as sterile water, physiological saline, glucose or the like.

The compositions of the present invention are administered in a manner compatible with the dosage formulation, and in a therapeutically effective amount. The quantity to be administered depends, for instance, on the subject and disease to be treated, capacity of the subject's organ, cellular and immune system to accommodate the therapeutic composition, and the nature of the cell or tissue therapy, etc. Precise amounts of therapeutic composition required to be administered depend on the judgment of the practitioner and are peculiar to each individual. Suitable regimes for initial administration and follow-on administration are also variable but can include an initial administration followed by repeated doses at one or more hour, or day, intervals by a subsequent injection or other administration.

As used herein, the terms “subject” and “patient” are used interchangeably and refer to an animal, including mammals such as non-primates (e.g., cows, pigs, horses, cats, dogs, rats etc.) and primates (e.g., monkey and human).

All the herein mentioned cells or cell populations (or cells derived therefrom) or all the herein mentioned compositions may be for medical use and/or for use in the treatment of any of the disorders and diseases herein indicated or for use in regenerative medicine, or for use as blood cells for transfusions.

As used herein, “treatment” refers to clinical intervention in an attempt to alter the disease course of the individual or cell being treated. Therapeutic effects of treatment include without limitation, preventing recurrence of disease, alleviation of symptoms, diminishment of any direct or indirect pathological consequences of the disease, decreasing the rate of disease progression, amelioration or palliation of the disease state, and remission or improved prognosis. As used herein, the terms “therapeutically effective amount” and “effective amount” are used interchangeably to refer to an amount of a composition of the invention that is sufficient to treat the immunological condition.

In some embodiments, a single administration of cells is provided. In other embodiments, multiple administrations are used. Multiple administrations can be provided over periodic time periods such as an initial treatment regime of 3 to 7 consecutive days, and then repeated at other times.

Further contemplated are methods involving co-administration, that is, administration of a composition of the invention before, after, or contemporaneously with administration of a treatment that may deplete the immune system or immune response of a subject.

Aspects described herein stem from, at least in part, development of methods that efficiently direct differentiation of pluripotent stem cells (PSCs) into hematopoietic cells, including progenitors. In some embodiments, the culturing process of the invention may involve multiple differentiation stages (e.g., 2, 3, or more). In some embodiment, the total time period for the in vitro or ex vivo culturing process described herein can range from about 6-14 days (e.g., 7-13 days, 7-12 days, or 8-11 days). In one example, the total time period is about 8 days.

In some embodiments, the methods for producing hematopoietic progenitors as disclosed herein may include multiple differentiation stages (e.g., 2, 3, 4, or more). For example, a mesoderm differentiation step, e.g., the culturing of the pluripotent stem cells under differentiation conditions to obtain cells of the mesoderm, a hematopoietic specification step, e.g., the culturing of the obtained mesoderm cells under differentiation conditions to obtain the hematopoietic progenitor cells. In some aspects, the present disclosure includes additional differentiation stages, for example a erythroid maturation step, a myeloid maturation step and/or a lymphoid maturation step.

The mesoderm differentiation medium is e.g. a medium able to differentiate PSCs in hematopoietic mesoderm, e.g. of such media are StemPro34, X-Vivo, StemSpan, APEL as well as B27- and/or N2-based media containing various combinations of cytokines or chemical modulators of BMP4, FGF, Activin/TGFb, WNT and VEGF signaling.

The hematovascular differentiation medium is e.g. a medium able to differentiate PSCs in hematopoietic and vascular cells using e.g. cytokines or chemical modulators of BMP4, FGF, Activin/TGFb, WNT, VEGF, SCF and Interleukins signaling.

The in vitro or ex vivo model described herein can provide a reliable source of hematopoietic progenitor cells. The PSC-derived hematopoietic progenitors can be used in various applications, including, e.g., but not limited to, as an in vitro model for hematopoiesis, related diseases or disorders, drug discovery and/or developments.

Accordingly, the present invention or embodiments of various aspects described herein relate to methods for generation of hematopoietic progenitor cells from PSCs, hematopoietic cells from PSCs, cells produced or derived by the same, and methods of use.

The in vitro or ex vivo method disclosed herein may use pluripotent stem cells (e.g., human pluripotent stem cells) or totipotent stem cells (e.g., human blastocysts or blastoids) as the starting material for producing hematopoietic cells. As used herein, “pluripotent” or “pluripotency” refers to the potential to form all types of specialized cells of the three germ layers (endoderm, mesoderm, and ectoderm); and is to be distinguished from “totipotent” or “totipotency”, that is the ability to form a complete embryo capable of giving rise to offsprings. As used herein, “human pluripotent stem” (hPS) cells refers to human cells that have the capacity, under appropriate conditions, to self-renew as well as the ability to form any type of specialized cells of the three germ layers (endoderm, mesoderm, and ectoderm). hPS cells may have the ability to form a teratoma in 8-12 week old SCID mice and/or the ability to form identifiable cells of all three germ layers in tissue culture. Included in the definition of human pluripotent stem cells are embryonic cells of various types including human embryonic stem (hES) cells, (see, e.g., Thomson et al. (1998), Heins et. al. (2004), as well as induced pluripotent stem cells [see, e.g. Takahashi et al., (2007); Zhou et al. (2009); Yu and Thomson in Essentials of Stem Cell Biology (2nd Edition]. The various methods described herein may utilize hPS cells from a variety of sources. For example, hPS cells suitable for use may have been obtained from developing embryos by use of a nondestructive technique such as by employing the single blastomere removal technique described in e.g. Chung et al (2008), further described by Mercader et al. in Essential Stem Cell Methods (First Edition, 2009). Additionally or alternatively, suitable hPS cells may be obtained from established cell lines or may be adult stem cells.

Various techniques for obtaining hES cells are known to those skilled in the art. In some instances, the hES cells for use according to the present disclosure are ones, which have been derived (or obtained) without destruction of the human embryo, such as by employing the single blastomere removal technique known in the art. See, e.g., Chung et al., Cell Stem Cell, 2(2): 1 13-1 17 (2008), Mercader et al., Essential Stem Cell Methods (First Edition, 2009). Suitable hES cell lines can also be used in the methods disclosed herein. Examples include, but are not limited to, cell lines H1, H9, SA167, SA181, SA461 (Cellartis AB, Goteborg, Sweden) which are listed in the NIH stem cell registry, the UK Stem Cell bank and the European hESC registry and are available on request. Other suitable cell lines for use include those established by Klimanskaya et al., Nature 444:481-485 (2006), such as cell lines MA01 and MA09, and Chung et al., Cell Stem Cell, 2(2):1 13-1 17 (2008), such as cell lines MA126, MA127, MA128 and MA129, which all are listed with the International Stem Cell Registry (assigned to Advanced Cell Technology, Inc. Worcester, MA, USA).

Alternatively, the pluripotent stem cells for use in the methods disclosed herein may be induced pluripotent stem cells (iPS) cells such as human PS cells (hiPS cells). hiPS cells are a type of pluripotent stem cells derived from non-pluripotent cells—typically adult somatic cells—by induction of the expression of genes associated with pluripotency, such as SSEA-3, SSEA-4, TRA-1-60, TRA-1-81, Oct-4, Sox2, Nanog and Lin28. Various techniques for obtaining such PS cells have been established and all can be used in the present disclosure. See, e.g., Takahashi et al., Cell 131 (5):861-872 (2007); Zhou et al., Cell Stem Cell. 4(5):381-384 (2009); Yu and Thomson in Essentials of Stem Cell Biology (2nd Edition, Chapter 4)].

The hematopoietic progenitor cells may also be derived from other pluripotent stem cells such as adult stem cells, cancer stem cells or from other embryonic, fetal, juvenile or adult sources. Human pluripotent stem cells, (wherein hPS cells can comprise both human embryonic stem cells (hES) cells and human induced pluripotent stem cells (hiPS) cells) can be cultured until about 70% confluence. These cells can be removed from these conditions, dissociated into clumps (termed “embryoid bodies”), and then further cultured under hypoxic conditions (about 5% O₂, 5% CO₂) in defined serum-free differentiation media.

Preferably the human pluripotent stem cells are not derived from human embryo and/or are not human embryonic stem cells (hES) cells.

In some embodiments, ES cell culture may be grown on one layer of feeder cells. “Feeder cells” refer to a type of cell, which can be second species, when being co-cultured with another type of cell. Feeder cells are generally derived from embryo tissue or tire tissue fibroblast. Embryo is collected from the CF1 mouse of pregnancy 13 days, is transferred in 2 ml trypsase/EDTA, then careful chopping, 37 DEG C incubate 5 minutes. 10% FBS is added, so that fragment is precipitated, cell increases in 90% DMEM, 10% FBS and 2 mM glutamine. The feeder cells offer a growing environment for the ES cells. Certain form of ES cells can use, for example, primary mouse embryonic fibroblast or infinite multiplication mouse embryonic fibroblasts. In order to prepare feeder layer, irradiated cells may be used to support the ES cells (about 3000 rad g-radiation will inhibit proliferation).

In some embodiments, the PS cells are removed from the feeder cells and cultured in serum free defined media for about 24 hours to generate embryoid bodies. Term “embryoid” is synonymous with “aggregation”, refers to differentiated and neoblast aggregation, which appears in ES cells. It is maintained in undue growth or the culture that suspends in monolayer cultures. Embryoid is different cell types (generally originating from different germinal layers) Mixture, can according to morphological criteria distinguish and available immunocytochemistry detect cell marking. In some embodiments, the PS cells are cultured in a cell culture vessel coated with at least one extracellular matrix protein (e.g., laminin or Matrigel) to generate embryoid bodies.

The methods herein disclosed may involve a step of differentiation to differentiate the PS cells disclosed herein to hematopoietic progenitor cells.

Suitable conditions for mesoderm differentiation are known in the art (e.g., Sturgeon et al., Nat Biotechnol.; 32(6):554-61 (2014)) and/or disclosed in Examples below. As used herein “mesoderm” and “mesoderm cells (ME cells)” refers to cells exhibiting protein and/or gene expression as well as morphology typical to cells of the mesoderm or a composition comprising a significant number of cells resembling the cells of the mesoderm. The mesoderm is one of the three germinal layers that appears in the third week of embryonic development. It is formed through a process called gastrulation. There are three important components, the paraxial mesoderm, the intermediate mesoderm and the lateral plate mesoderm. The paraxial mesoderm forms the somitomeres, which give rise to mesenchyme of the head and organize into somites in occipital and caudal segments, and give rise to sclerotomes (cartilage and bone), and dermatomes (subcutaneous tissue of the skin). Signals for somite differentiation are derived from surroundings structures, including the notochord, neural tube and epidermis. The intermediate mesoderm connects the paraxial mesoderm with the lateral plate, eventually it differentiates into urogenital structures consisting of the kidneys, gonads, their associated ducts, and the adrenal glands. The lateral plate mesoderm give rise to the heart, blood vessels and blood cells of the circulatory system as well as to the mesodermal components of the limbs.

Some of the mesoderm derivatives include the muscle (smooth, cardiac and skeletal), the muscles of the tongue (occipital somites), the pharyngeal arches muscle (muscles of mastication, muscles of facial expressions), connective tissue, dermis and subcutaneous layer of the skin, bone and cartilage, dura mater, endothelium of blood vessels, red blood cells, white blood cells, and microglia, the kidneys and the adrenal cortex.

Generally, in order to obtain ME cells, PS cells such as hPS cells can be cultured in a differentiation medium comprising L-glutamine, ascorbic acid, monothioglycerol, and a differentiation inducer such as transferrin. The differentiation medium may be optionally further supplemented with one or more growth factors, such as a fibroblast growth factor (FGF) (e.g., FGF1, FGF2 and FGF4), and one or more bone morphogenic proteins (BMP), such as BMP2 and BMP4. As used herein, the term “FGF” means fibroblast growth factor, preferably of human and/or recombinant origin, and subtypes belonging thereto are e.g. “bFGF” (means basic fibroblast growth factor, sometimes also referred to as FGF2) and FGF4. “aFGF” means acidic fibroblast growth factor (sometimes also referred to as FGF1). As used herein, the term “BMP” means Bone Morphogenic Protein, preferably of human and/or recombinant origin, and subtypes belonging thereto are e.g. BMP4 and BMP2. The concentration of the one or more growth factors may vary depending on the particular compound used. The concentration of FGF2, for example, is usually in the range of about 2 to about 50 ng/ml, such as about 2 to about 20 ng/ml. FGF2 may, for example, be present in the specification medium at a concentration of 9 or 10 ng/ml. The concentration of FGF1, for example, is usually in the range of about 50 to about 200 ng/ml, such as about 80 to about 120 ng/ml. FGF1 may, for example, be present in the specification medium at a concentration of about 100 ng/ml. The concentration of FGF4, for example, is usually in the range of about 20 to about 40 ng/ml. FGF4 may, for example, be present in the specification medium at a concentration of about 30 ng/ml. The concentration of the one or more BMPs, is usually in the range of about 50 to about 300 ng/ml, such as about 50 to about 250 ng/ml, about 100 to about 250 ng/ml, about 150 to about 250 ng/ml, about 50 to about 200 ng/ml, about 100 to about 200 ng/ml or about 150 to about 200 ng/ml. The concentration of BMP2, for example, is usually in the range of about 2 to about 50 ng/ml, such as about 10 to about 30 ng/ml. BMP2 may, for example, be present in the hepatic specification medium at a concentration of about 20 ng/ml.

In one aspect, from about days 0-3 of differentiation, embryoid bodies can be exposed to recombinant human BMP4. On about days 1-3 of differentiation, bFGF can be added to the differentiation media.

In some embodiments, the differentiation media comprises an activin, such as activin A or B. The concentration of activin is usually in the range of about 50 to about 200 ng/ml, such as about 80 to about 120 ng/ml. Activin may, for example, be present in the differentiation medium at a concentration of about 90 ng/ml or about 100 ng/ml. As used herein, the term “Activin” is intended to mean a TGF-beta family member that exhibits a wide range of biological activities including regulation of cellular proliferation and differentiation such as “Activin A” or “Activin B”. Activin belongs to the common TGF-beta superfamily of ligands. The differentiation medium may further comprise an inhibitor of the activin receptor-like kinase receptors, ALK5, ALK4 and ALK7, such as SB431542. The concentration of the ALK5, ALK4 and ALK7 inhibitor is usually in the concentration of about 1 mM to about 12 mM, such as about 3 mM to about 9 mM. The differentiation media may comprise a GSKp-inhibitor, such as, e.g., CHIR99021 or CHIR98014, or an activator of WNT signaling, such as WNT3A.

The concentration of the activator of WNT signaling is usually in the range of about 0.05 to about 90 ng/ml, such as about 50 ng/ml. As used herein, “activator of WNT signaling” refers to a compound which activates WNT signaling. The concentration of the GSKp inhibitor, if present, is usually in the range of about 0.1 to about 10 mM, such as about 0.05 to about 5 mM.

The concentration of serum, if present, is usually in the range of about 0.1 to about 2% v/v, such as about 0.1 to about 0.5%, about 0.2 to about 1.5% v/v, about 0.2 to about 1% v/v, about 0.5 to 1% v/v or about 0.5 to about 1.5% v/v. Serum may, for example, if present, in the differentiation medium may be at a concentration of about 0.2% v/v, about 0.5% v/v or about 1% v/v. In one aspect, the differentiation medium omits serum and instead comprises a suitable serum replacement.

The culture medium forming the basis for the differentiation medium may be any culture medium suitable for culturing PS cells and is not particularly limited. For example, base media such as StemPro-34 media, RPMI 1640 or advanced medium, Dulbecco's Modified Eagle Medium (DMEM), Iscove's Modified Dulbecco's Media (IMDM) F-12 Medium (also known as Ham's F-12), or MEM may be used. Thus, the differentiation medium may be StemPro-34 media or advanced medium comprising or supplemented with the above-mentioned components. In some embodiments, the base media may be a blend of two or more suitable culture medias, for example, the base media may be a blend of IMDM and F-12. In some embodiments, the differentiation medium may be DMEM or a blend comprising DMEM comprising or supplemented with the above-mentioned components. The differentiation medium may thus also be MEM medium or a blend comprising MEM comprising or supplemented with the above-mentioned components. In some embodiments, the differentiation medium may be IMDM or a blend comprising IMDM comprising or supplemented with the above-mentioned components. In some embodiments, the differentiation medium may be F-12 or a blend comprising F-12 comprising or supplemented with the above-mentioned components.

In some embodiments, the differentiation medium comprises, consists essentially of, or consists of, a base medium supplemented with L-glutamine, ascorbic acid, monothioglycerol, transferrin and BMP-4. In other embodiments, the differentiation medium comprises, consists essentially of, or consists of, a base medium supplemented with L-glutamine, ascorbic acid, monothioglycerol, transferrin, BMP-4 and bFGF. In still other embodiments, the differentiation medium comprises, consists essentially of, or consists of, a base medium supplemented with L-glutamine, ascorbic acid, monothioglycerol, transferrin, BMP-4, bFGF, an ALK5, ALK4 and ALK7 inhibitor, and a GSKp-inhibitor. In another embodiment, the differentiation medium comprises, consists essentially of, or consists of a base medium, 2 mM L-glutamine, 1 mM ascorbic acid, monothioglycerol, 150 mg/mL transferrin and BMP-4. In yet another embodiment, the differentiation medium comprises, consists essentially of, or consists of, a base medium, 2 mM L-glutamine, 1 mM ascorbic acid, monothioglycerol, 150 mg/mL transferrin, BMP-4 and 5 ng/mL bFGF. In still yet another embodiment, the differentiation medium comprises, consists essentially of, or consists of, a base medium, 2 mM L-glutamine, 1 mM ascorbic acid, monothioglycerol, 150 mg/mL transferrin, BMP-4 and 5 ng/mL bFGF, 6 mM SB431542, and 3 mM CHIR99021. PS cells are normally cultured for up to 3-4 days in suitable differentiation medium in order to obtain mesoderm cells. For example, from about days 0-3 of differentiation, embryoid bodies can be exposed to recombinant human BMP4.

On about days 1-3 of differentiation, bFGF can be added to the differentiation media.

On day 2, fresh media can be replaced, with the addition of a WNT signaling stimulating agent (a GSK3b antagonist or inhibitor, such as CHIR99021 or analogs thereof, such as CHIR98014; a recombinant WNT protein; or a WNT agonist) and ACTIVIN/NODAL signaling suppressing agent (e.g., an ALK inhibitor, such as SB-431542 or a small molecule TGFb inhibitor). In some embodiments, the PS cells are cultured in a cell culture vessel coated with at least one extracellular matrix protein (e.g., laminin or Matrigel) during contact with the differentiation medium. The PS cells may be dissociated and collected in suspension (e.g., through contact with TrypLE), if needed.

Following the mesoderm differentiation step, the obtained mesoderm cells are isolated for the presence of CD32 and can be further cultured in a hematopoietic progenitor specification medium to obtain hematopoietic progenitor cells.

In the present method the cultivating or differentiation and isolation steps may be performed in sequence or simultaneously. For example, the cultivation or differentiation step may be performed before (or after) the isolation step or they may be performed at the same time.

As used herein, “induced pluripotent stem cells” or “iPSCs” are cells that can be generated from somatic cells by genetic manipulation of somatic cells, e.g., by retroviral transduction of somatic cells such as fibroblasts, hepatocytes, gastric epithelial cells with transcription factors such as Oct-3/4, Sox2, c-Myc, and KLF4 [Yamanaka S, Cell Stem Cell. 2007, 1 (1):39-49; Aoi T, et al., Generation of Pluripotent Stem Cells from Adult Mouse Liver and Stomach Cells. Science. 2008 Feb. 14. (Epub ahead of print); I H Park, Zhao R, West J A, et al. Reprogramming of human somatic cells to pluripotency with defined factors. Nature 2008; 451:141-146; K Takahashi, Tanabe K, Ohnuki M, et al. Induction of pluripotent stem cells from adult human fibroblasts by defined factors. Cell 2007; 131:861-872], Other embryonic-like stem cells can be generated by nuclear transfer to oocytes, fusion with embryonic stem cells or nuclear transfer into zygotes if the recipient cells are arrested in mitosis. In addition, iPSCs may be generated using non-integrating methods e.g., by using small molecules or RNA.

The term “embryonic stem cells” refers to embryonic cells that are capable of differentiating into cells of all three embryonic germ layers (i.e., endoderm, ectoderm and mesoderm), or remaining in an undifferentiated state. The term “embryonic stem cells” may comprise cells which are obtained from the embryonic tissue formed after gestation (e.g., blastocyst) before implantation of the embryo (i.e., a pre-implantation blastocyst), extended blastocyst cells (EBCs) which are obtained from a post-implantation/pre-gastrulation stage blastocyst (see WO 2006/040763) and embryonic germ (EG) cells which are obtained from the genital tissue of a foetus any time during gestation, preferably before 10 weeks of gestation. In embodiments, embryonic stem cells are obtained using well-known cell-culture methods. For example, human embryonic stem cells can be isolated from human blastocysts. Commercially available stem cells can also be used in aspects and embodiments of the present disclosure. Human ES cells may be purchased from the NIH human embryonic stem cells registry, www.grants.nih.govstem_cells/ or from other hESC registries. Non-limiting examples of commercially available embryonic stem cell lines are HAD-C 102, ESI, BGO 1, BGO2, BGO3, BGO4, CY12, CY30, CY92, CY1O, TE03, TE32, CHB-4, CHB-5, CHB-6, CHB-8, CHB-9, CHB-10, CHB-11, CHB-12, HUES 1, HUES 2, HUES 3, HUES 4, HUES 5, HUES 6, HUES 7, HUES 8, HUES 9, HUES 10, HUES 11, HUES 12, HUES 13, HUES 14, HUES 15, HUES 16, HUES 17, HUES 18, HUES 19, HUES 20, HUES 21, HUES 22, HUES 23, HUES 24, HUES 25, HUES 26, HUES 27, HUES 28, CyT49, RUES3, WAO 1, UCSF4, NYUES 1, NYUES2, NYUES3, NYUES4, NYUESS, NYUES6, NYUES7, UCLA 1, UCLA 2, UCLA 3, WA077 (H7), WA09 (H9), WA 13 (HI 3), WA14 (H14), HUES 62, HUES 63, HUES 64, CT I, CT2, CT3, CT4, MA135, Eneavour-2, WIBR 1, WIBR2, WIBR3, WIBR4, WIBRS, WIBR6, HUES 45, Shef 3, Shef 6, BINheml9, BJNhem20, SAGO 1, SAOO1.

ES cells can also be obtained from other species, including mouse (Mills and Bradley, 2001), golden hamster [Doetschman et al., 1988, Dev Biol. 127: 224-7], rat [Iannaccone et al., 1994, Dev Biol. 163: 288-92], rabbit [Giles et al. 1993, Mol Reprod Dev. 36: 130-8; Graves & Moreadith, 1993, Mol Reprod Dev. 1993, 30 36: 424-33], several domestic animal species [Notarianni et al., 1991, J Reprod Fertil Suppl. 43: 255-60; Wheeler 1994, Reprod Fertil Dev. 6: 563-8; Mitalipova et al., 2001, Cloning. 3: 59-67] and non-human primate species (Rhesus monkey and marmoset) [Thomson et al., 1995, Proc Natl Acad Sci USA. 92: 7844-8; Thomson et al., 1996, Biol Reprod. 55: 254-9].

Extended blastocyst cells (EBCs) can be obtained from a blastocyst of at least nine days post fertilization at a stage prior to gastrulation. Prior to culturing the blastocyst, the zona pellucida may be digested [for example by Tyrode's acidic solution (Sigma Aldrich, St Louis, MO, USA)] so as to expose the inner cell mass. The blastocysts are then cultured as whole embryos for at least nine and no more than fourteen days post fertilization (i.e., prior to the gastrulation event) in vitro using standard embryonic stem cell culturing methods.

Another method for preparing ES cells is described in Chung et al., Cell Stem Cell, Volume 2, Issue 2, 113-117, 7 Feb. 2008. This method comprises removing a single cell from an embryo during an in vitro fertilization process. The embryo is not destroyed in this process.

EG (embryonic germ) cells may be prepared from the primordial germ cells obtained from fetuses of about 8-11 weeks of gestation (in the case of a human foetus) using laboratory techniques known to anyone skilled in the arts. The genital ridges are dissociated and cut into small portions which are thereafter disaggregated into cells by mechanical dissociation. The EG cells are then grown in tissue culture flasks with the appropriate medium. The cells are cultured with daily replacement of medium until a cell morphology consistent with EG cells is observed, typically after 7-30 days or 1-4 passages. For additional details on methods of preparation human EG cells see Shamblott et al., [Proc. Natl. Acad. Sci. USA 95: 13726, 1998] and U.S. Pat. No. 6,090,622. A further method for preparing ES cells is by parthenogenesis. The embryo is also not destroyed in the process.

As used herein, “hematopoietic progenitors” or “hematopoietic stem cells” may mean definitive hematopoietic stem cells that are capable of engrafting a recipient of any age post-birth. As described above, hematopoietic progenitors can be derived from: embryonic tissue, embryonic stem cells (ESC), induced pluripotent stem cells (iPSC), or reprogrammed cells of other types (non-pluripotent cells of any type reprogrammed into HSCs). The hematopoietic progenitor cells may be non-fetal liver HSC, adult peripheral blood HSC or umbilical cord blood HSC.

“Hematopoietic progenitors” may generally be characterized, and thus identified, by one or more of a gene or protein expression of CD34+CD43^{neg}CD73^{neg}CD184^{neg}. The hematopoietic progenitor cells can be a hemogenic endothelial (HE) population that is capable of multi-lineage definitive hematopoiesis, at a clonal level.

The term “Hematopoietic progenitors” and “Hematopoietic progenitor cells” have the same meaning and may be used interchangeably.

In general, in order to obtain hematopoietic progenitor cells, mesoderm cells, for example, mesoderm cells as described above, are further cultured in a hematopoietic differentiation medium comprising one or more growth factors, such as a fibroblast growth factor (FGF) (e.g., FGF1, FGF2 and FGF4) and one or more vascular endothelial growth factor (VEGF). The concentration of the one or more growth factors may vary depending on the particular compound used. The concentration of bFGF, for example, is usually in the range of about 1 to about 10 ng/ml, such as about 2 to about 8 ng/ml. bFGF may, for example, be present in the specification medium at a concentration of 3 or 7 ng/ml. The concentration of VEGF, for example, is usually in the range of about 2 to about 50 ng/ml, such as about 2 to about 20 ng/ml. VEGF may, for example, be present in the specification medium at a concentration of 9 or 15 ng/ml.

The specification medium may include other factors such as stem cell factor (SCF), Interleukin-6, 3, and 11, insulin growth factors such as IGF-1, and erythropoietin (EPO). SCF, when present, is included at a concentration between about 1 to about 100 ng/ml, such as about 20 to about 80 ng/ml. Interleukin when present, is included at a concentration between about 1 ng/mL to about 20 ng/mL, such as about 5 ng/ml to about 10 ng/ml. EPO, when present, is included at a concentration between about 1 U/mL to about 5 U/mL.

In some embodiments, the specification medium comprises, consists essentially of, or consists of, a base medium supplemented with a fibroblast growth factor, a vascular endothelial growth factor (VEGF). In another embodiment, the specification medium comprises, consists essentially of, or consists of a base medium, 5 ng/mL bFGF and 15 ng/mL VEGF. In another aspect, the specification medium consists essentially of, or consists of, a base medium supplemented with IL-6, IGF-1, SCF and EPO. In another aspect, the specification medium consists essentially of, or consists of, a base medium supplemented with 10 ng/mL IL-6, 25 ng/ml IGF-1, 50 ng/mL SCF and 2 U/mL EPO

The culture medium forming the basis for the hematopoietic specification medium may be any culture medium suitable for culturing mesodermal cells and is not particularly limited. For example, the culture medium forming the basis for the specification medium may be any culture medium suitable for culturing ME cells and is not particularly limited. For example, base media such as StemPro-34 media, RPMI 1640 or advanced medium, Dulbecco's Modified Eagle Medium (DMEM), Iscove's Modified Dulbecco's Media (IMDM) F-12 Medium (also known as Ham's F-12), or MEM may be used. Thus, the differentiation medium may be StemPro-34 media or advanced medium comprising or supplemented with the above-mentioned components. In some embodiments, the base media may be a blend of two or more suitable culture medias, for example, the base media may be a blend of IMDM and F-12. In some embodiments, the differentiation medium may be DMEM or a blend comprising DMEM comprising or supplemented with the above-mentioned components. The differentiation medium may thus also be MEM medium or a blend comprising MEM comprising or supplemented with the above-mentioned components. In some embodiments, the differentiation medium may be IMDM or a blend comprising IMDM comprising or supplemented with the above-mentioned components. In some embodiments, the differentiation medium may be F-12 or a blend comprising F-12 comprising or supplemented with the above-mentioned components. In some embodiments, the ME cells are cultured in a cell culture vessel coated with at least one extracellular matrix protein (e.g., laminin) during contact with the hematopoietic specification medium.

For specification into hematopoietic progenitor cells, ME cells are normally cultured for up to 3 days in specification medium comprising bFGF and VEGF. The ME cells may then, for example, be cultured in a specification medium comprising IL-6, IGF-1, IL-11, SCF and EPO for an additional 2 days to about 5 days. In some embodiments, the ME cells are maintained in the cell culture vessel optionally coated with at least one extracellular matrix protein, during specification to hematopoietic progenitor cells.

BMP4, then bFGF, then WNT, and ACTIVIN/NODAL, followed by VEGF can be used to derive different population of progenitors from embryonic stem cells and induced pluripotent stem cells (collectively, human pluripotent stem cells, hPSCs).

The hematopoietic progenitor cells obtained from the hematopoietic specification step may be further cultured in a maturation medium to be differentiated into specific types of blood cells (e.g., red blood cells, platelets, neutrophils, megakaryocytes, etc.) in vitro or ex vivo before administration to a subject. The hematopoietic progenitor cells can be differentiated into specific types of blood cells using any methods described herein or known in the art. For example, any of the growth factors known to promote cell differentiation into specific types of hematopoietic cells described herein or known in the art can be used. In particular, the following references describe methods for differentiation of hematopoietic progenitor cells that can be used for differentiation of the hematopoietic progenitor cells: Zeuner et al., 2012, Stem Cells 30:1587-96; Ebihara et al., 2012, Int J Hematol 95:610-6; Takayama & Eto, 2012, Cell Mol Life Sci 69:3419-28; Takayama & Eto, 2012, Methods Mol Biol 788:205-17; and Kimbrel & Lu, 2011, Stem Cells Int., March 8; doi: 10.4061/2011/273076. In one embodiment, the hematopoietic progenitor cells are differentiated into red blood cells; such red blood cells can be administered to a subject. In one embodiment, the hematopoietic progenitor cells are differentiated into neutrophils; and such neutrophils can be administered to a subject. In one embodiment, the hematopoietic progenitor cells are differentiated into platelets; and such platelets can be administered to a patient. In certain embodiments, hematopoietic progenitor cells are generated in accordance with the methods described herein (optionally, gene-corrected), differentiated into specific types of hematopoietic cells (e.g., red blood cells, neutrophils or platelets), and the differentiated cells produced from the hematopoietic progenitor cells are administered to a subject.

Methods and products as described herein with respect to the hematopoietic progenitor cells will also apply to the differentiated cells produced from the hematopoietic progenitor cells, unless the context would indicate otherwise to one skilled in the art.

The pluripotent stem cells or the hematopoietic progenitor or the hematopoietic cell or the HEC of the invention may be genetically modified such that a gene of interest is modulated.

In the present invention are comprised methods of preparing such genetically modified pluripotent stem cells or hematopoietic progenitor cells. In some embodiments, the gene of interest is disrupted. As used herein, the term “a disrupted gene” refers to a gene containing one or more mutations (e.g., insertion, deletion, or nucleotide substitution, etc.) relative to the wild-type counterpart so as to substantially reduce or completely eliminate the activity of the encoded gene product. The one or more mutations may be located in a non-coding region, for example, a promoter region, a regulatory region that regulates transcription or translation; or an intron region.

Alternatively, the one or more mutations may be located in a coding region (e.g., in an exon). In some instances, the disrupted gene does not express or express a substantially reduced level of the encoded protein. In other instances, the disrupted gene expresses the encoded protein in a mutated form, which is either not functional or has substantially reduced activity. In some embodiments, a disrupted gene does not express (e.g., encode) a functional protein.

Techniques such as CRISPR (particularly using Cas9 and guide RNA), editing with zinc finger nucleases (ZFNs) and transcription activator-like effector nucleases (TALENs) may be used to produce the genetically engineered pluripotent stem cells. ‘Genetic modification’, ‘genome editing’, or ‘genomic editing’, or ‘genetic editing’, as used interchangeably herein, is a type of genetic engineering in which DNA is inserted, deleted, and/or replaced in the genome of a targeted cell.

Targeted genome modification (interchangeable with “targeted genomic editing” or “targeted genetic editing”) enables insertion, deletion, and/or substitution at pre-selected sites in the genome. When an endogenous sequence is deleted at the insertion site during targeted editing, an endogenous gene comprising the affected sequence may be knocked-out or knocked-down due to the sequence deletion. In another aspect, an endogenous gene may be modified by introducing a change in an endogenous gene codon, wherein the modification introduces an amino acid change in the gene product or introduction of a stop codon. Therefore, targeted modification may also be used to disrupt endogenous gene expression with precision. Similarly used herein is the term “targeted integration,” referring to a process involving insertion of one or more exogenous sequences, with or without deletion of an endogenous sequence at the insertion site. In comparison, randomly integrated genes are subject to position effects and silencing, making their expression unreliable and unpredictable. For example, centromeres and sub-telomeric regions are particularly prone to transgene silencing. Reciprocally, newly integrated genes may affect the surrounding endogenous genes and chromatin, potentially altering cell behavior or favoring cellular transformation. Therefore, inserting exogenous DNA in a pre-selected locus such as a safe harbor locus, or genomic safe harbor (GSH) is important for safety, efficiency, copy number control, and for reliable gene response control.

Targeted modification can be achieved either through a nuclease-independent approach, or through a nuclease-dependent approach. In the nuclease-independent targeted editing approach, homologous recombination is guided by homologous sequences flanking an exogenous polynucleotide to be inserted, through the enzymatic machinery of the host cell. Alternatively, targeted modification could be achieved with higher frequency through specific introduction of double strand breaks (DSBs) by specific rare-cutting endonucleases. Such nuclease-dependent targeted editing utilizes DNA repair mechanisms including non-homologous end joining (NHEJ), which occurs in response to DSBs. Without a donor vector containing exogenous genetic material, the NHEJ often leads to random insertions or deletions (in/dels) of a small number of endogenous nucleotides. In comparison, when a donor vector containing exogenous genetic material flanked by a pair of homology arms is present, the exogenous genetic material can be introduced into the genome during homology directed repair (HDR) by homologous recombination, resulting in a “targeted integration.”

In some embodiments, the hematopoietic progenitor cells are obtained from cells derived from a subject to whom the hematopoietic progenitor cells are to be administered. In such embodiments, the embryonic hematopoietic stem cells can be derived from ESC, iPSC or reprogrammed non-pluripotent cells derived from the subject to whom the hematopoietic progenitor cells or cells derived therefrom are to be administered. In a specific embodiment, adult cells can be obtained from a subject, such cells can be reprogrammed to iPSC and then hematopoietic progenitor cells of the disclosure. In specific embodiments, hematopoietic progenitor cells are derived from cells of a patient with a genetic disorder associated with a gene having a sequence detect, and such hematopoietic progenitor cells are genetically engineered to correct the sequence defect before administration to the subject. In one embodiment, hematopoietic progenitor cells are derived from cells of a subject with a genetic disorder associated with a gene having a sequence defect, and such hematopoietic progenitor cells are genetically engineered to correct the sequence defect, and the genetically engineered hematopoietic progenitor cells or cells derived therefrom are administered to the patient.

Once generated the hematopoietic progenitor cells or cells differentiated therefrom can be cryopreserved in accordance with the methods described below or known in the art.

The hematopoietic progenitor cells, whether recombinantly expressing a desired gene, having been corrected for a defective gene, or not, can be administered into a human subject in need thereof for hematopoietic function for the treatment of disease or injury or for gene therapy by any method known in the art which is appropriate for the hematopoietic progenitor cells and the transplant site. Preferably, the hematopoietic progenitor cells or cells derived therefrom are transplanted (infused) intravenously. In one embodiment, the hematopoietic progenitor cells differentiate into cells of the myeloid lineage in the patient. In another embodiment, the hematopoietic progenitor cells differentiate into cells of the lymphoid lineage in the patient.

In one embodiment, the transplantation of the hematopoietic progenitor cells is autologous. In such embodiments, before expansion, cells are isolated from tissues of a subject to whom hematopoietic progenitor cells are to be administered, reprogrammed to iPSC and then hematopoietic progenitor cells, or directly reprogrammed to hematopoietic progenitor cells and, optionally, gene-corrected as described above. In other embodiments, the transplantation of the hematopoietic progenitor cells is non-autologous. In some of these embodiments, the transplantation of the hematopoietic progenitor cells is allogeneic. For non-autologous transplantation, the recipient can be given an immunosuppressive drug to reduce the risk of rejection of the transplanted cells. In some embodiments, the transplantation of the hematopoietic progenitor cell is syngeneic.

In specific embodiments, hematopoietic progenitor cells or cells derived therefrom are administered to a subject with a hematopoietic disorder as described herein.

The hematopoietic progenitor cells populations can be administered by any convenient route, for example by infusion or bolus injection, and may be administered together with other biologically active agents. Administration can be systemic or local.

The titer of the hematopoietic progenitor cells administered which will be effective in the treatment of a particular disorder or condition will depend on the nature of the disorder or condition, and can be determined by standard clinical techniques. In addition, in vitro and in vivo assays may optionally be employed to help identify optimal dosage ranges. The precise dose to be employed in the formulation will also depend on the route of administration, and the seriousness of the disease or disorder, and should be decided according to the judgment of the practitioner and each subject's circumstances. In specific embodiments, suitable dosages of the hematopoietic progenitor cells for administration are generally about at least 5×10⁶, 10⁷, 5×10⁷, 75×10⁶, 10⁷, 5×10⁷, 10⁸, 5×10⁸, 1×10⁹, 5×10⁹, 1×10¹⁰, 5×10¹⁰, 1×10¹¹, 5×10¹¹ or 10¹² CD32+ cells per kilogram patient weight, and most preferably about 10⁷ to about 10¹² CD32+ cells per kilogram patient weight, and can be administered to a patient once, twice, three or more times with intervals as often as needed.

In certain embodiments, the patient is a human patient, preferably a human patient with a hematopoietic disorder or an immunodeficient human patient.

In a specific embodiment, the hematopoietic progenitor cell population administered to a human patient in need thereof can be a pool of two or more samples derived from a single human. As used herein the terms “patient” and “subject” are used interchangeably.

The present invention also includes methods of treatment by administration to a patient of a pharmaceutical (therapeutic) composition comprising a therapeutically effective amount of recombinant or non-recombinant hematopoietic progenitor cells produced by the methods of the present invention as described herein.

The present disclosure provides pharmaceutical compositions.

Such compositions comprise a therapeutically effective amount of the hematopoietic progenitor cells or cells derived therefrom, and a pharmaceutically acceptable carrier or excipient. Such a carrier can be but is not limited to saline, buffered saline, dextrose, water, glycerol, ethanol, and combinations thereof. The carrier and composition preferably are sterile. Suitable pharmaceutical carriers are described in Remington: The Science and Practice of Pharmacy, 21 st Edition, David B. Troy, ed., Lippicott Williams & Wilkins (2005), which is incorporated by reference herein in its entirety, and specifically for the material related to pharmaceutical carriers and compositions. The pharmaceutical compositions described herein can be formulated in any manner known in the art.

Hematopoietic progenitor cells can be resuspended in a pharmaceutically acceptable medium suitable for administration to a mammalian host. In preferred embodiments, the pharmaceutical composition is acceptable for therapeutic use in humans.

The pharmaceutical compositions described herein can be administered via any route known to one skilled in the art to be effective. In a preferred embodiment, the composition is formulated in accordance with routine procedures as a pharmaceutical composition adapted fir intravenous administration to a patient (e.g., a human). Typically, compositions for intravenous administration are solutions in sterile isotonic aqueous buffer. Where necessary, the composition may also include a solubilizing agent and a local anesthetic such as lidocaine to ease pain at the site of the injection.

In specific embodiments, the compositions described herein are formulated for administration to a patient with one or more additional therapeutic active ingredients.

The hematopoietic progenitor cells of the present disclosure can be used to provide hematopoietic function to a patient in need thereof, preferably a human patient. In other embodiments, the patient is a cow, a pig, a horse, a dog, a cat, or any other animal, preferably a mammal.

In one embodiment, administration of hematopoietic progenitor cells of the invention is for the treatment of immunodeficiency. In a preferred embodiment, administration of hematopoietic progenitor cells of the disclosure is for the treatment of pancytopenia or for the treatment of neutropenia. The immunodeficiency in the patient, for example, pancytopenia or neutropenia, can be the result of an intensive chemotherapy regimen, myeloablative regimen for hematopoietic cell transplantation (HCT), or exposure to acute ionizing radiation. Exemplary chemotherapeutics that can cause prolonged pancytopenia or prolonged neutropenia include, but are not limited to alkylating agents such as cisplatin, carboplatin, and oxaliplatin, mechlorethamine, cyclophosphamide, chlorambucil, and ifosfamide. Other chemotherapeutic agents that can cause prolonged pancytopenia or prolonged neutropenia include azathioprine, mercaptopurine, vinca alkaloids, e.g., vincristine, vinblastine, vinorelbine, vindesine, and taxanes. In particular, a chemotherapy regimen that can cause prolonged pancytopenia or prolonged neutropenia is the administration of clofarabine and Ara-C.

In one embodiment, the patient is in an acquired or induced aplastic state.

The immunodeficiency in the patient also can be caused by exposure to acute ionizing radiation following a nuclear attack, e.g., detonation of a “dirty” bomb in a densely populated area, or by exposure to ionizing radiation due to radiation leakage at a nuclear power plant, or exposure to a source of ionizing radiation, raw uranium ore.

Transplantation of hematopoietic cells or of hematopoietic progenitor cells of the invention can be used in the treatment or prevention of hematopoietic disorders and diseases or immunodeficiency disorders. In one embodiment, the hematopoietic progenitor cells are administered to a patient with a hematopoietic deficiency or an immunodeficiency. In one embodiment, the hematopoietic progenitor cells are used to treat or prevent a hematopoietic disorder or disease characterized by a failure or dysfunction of normal blood cell production and cell maturation. In another embodiment, the hematopoietic progenitor cells are used to treat or prevent a hematopoietic disorder or disease resulting from a hematopoietic malignancy. In yet another embodiment, the hematopoietic progenitor cells are used to treat or prevent a hematopoietic disorder or disease resulting from immunosuppression, particularly immunosuppression in subjects with malignant, solid tumors. In yet another embodiment, the hematopoietic progenitor cells are used to treat or prevent an autoimmune disease affecting the hematopoietic system or an immunodeficiency. In yet another embodiment, the hematopoietic progenitor cells are used to treat or prevent a genetic or congenital hematopoietic disorder or disease.

Examples of particular hematopoietic diseases and disorders which can be treated by the hematopoietic progenitor cells or hematopoietic cells of the disclosure include but are not limited to diseases resulting from a failure or dysfunction of normal blood cell production and maturation. In non-limiting examples, hyperproliferative stem cell disorders, aplastic anemia, pancytopenia, agranulocytosis, thrombocytopenia, red cell aplasia, Blackfan-Diamond syndrome, due to drugs, radiation, or infection Idiopathic II.

Hematopoietic malignancies, acute lymphoblastic (lymphocytic) leukemia, chronic lymphocytic leukemia, acute myelogenous leukemia, chronic myelogenous leukemia, acute malignant myelosclerosis, multiple myeloma polycythemia, vera angiogenic myelometaplasia, Waldenstrom's macroglobulinemia, Hodgkin's lymphoma, non-Hodgkin's lymphoma. Immunosuppression in patients with malignant, solid tumors, malignant melanoma, carcinoma of the stomach, ovarian carcinoma, breast carcinoma, small cell lung carcinoma, retinoblastoma, testicular carcinoma, glioblastoma, rhabdomyosarcoma, neuroblastoma, Ewing's sarcoma, lymphoma. Autoimmune diseases, rheumatoid arthritis, diabetes type I, chronic hepatitis, multiple sclerosis, systemic lupus, erythematosus. Genetic (congenital) disorders, anemias, familial aplastic Fanconi's syndrome (Fanconi anemia), Bloom's syndrome, pure red cell aplasia (PRCA), dyskeratosis, congenital Blackfan-Diamond syndrome, congenital dyserythropoietic syndromes. Shwachmann-Diamond syndrome, dihydrofolate reductase deficiencies, formamino transferase deficiency, Lesch-Nyhan syndrome, congenital spherocytosis, congenital elliptocytosis, congenital stomatocytosis, congenital Rh null disease, paroxysmal nocturnal hemoglobinuria, G6PD (glucose-6-phosphate dehydrogenase) variants, 1, 2, 3 pyruvate kinase deficiency, congenital erythropoietin sensitivity deficiency, sickle cell disease, and trait (Sickle cell anemia) thalassemia alpha, beta, gamma, met-hemoglobinemia, congenital disorders of immunity severe combined immunodeficiency disease (SCID), bare lymphocyte syndrome, ionophore-responsive combined immunodeficiency, combined immunodeficiency with a capping abnormality, nucleoside phosphorylase deficiency, granulocyte actin deficiency, infantile agranulocytosis, Gaucher's disease, adenosine deaminase deficiency, Kostmann's syndrome, reticular dysgenesis, congenital leukocyte dysfunction syndrome. Osteopetrosis, myelosclerosis, acquired hemolytic anemias, acquired immunodeficiencies infectious disorders causing primary or secondary immunodeficiencies bacterial infections (e.g., Brucellosis, Listerosis, tuberculosis, leprosy) parasitic infections (e.g., malaria, Leishmaniasis) fungal infections disorders involving disproportions in lymphoid cell sets and impaired immune functions due to aging phagocyte disorders Kostmann's agranulocytosis chronic granulomatous disease Chediak-Higachi syndrome neutrophil actin deficiency neutrophil membrane GP-180 deficiency metabolic storage diseases mucopolysaccharidoses mucolipidoses miscellaneous disorders involving immune mechanisms Wiskott-Aldrich Syndrome od-antitrypsin deficiency.

In one embodiment, the hematopoietic progenitor cells are administered to a patient with a hematopoietic deficiency. Hematopoietic deficiencies whose treatment with the hematopoietic progenitor cells of the disclosure is encompassed by the methods of the disclosure include but are not limited to decreased levels of either myeloid, erythroid, lymphoid, or megakaryocyte cells of the hematopoietic system or combinations thereof. In one embodiment, the hematopoietic progenitor cells are administered prenatally to a fetus diagnosed with hematopoietic deficiency. Among conditions susceptible to treatment with the hematopoietic progenitor cells of the present disclosure is leukopenia, a reduction in the number of circulating leukocytes (white cells) in the peripheral blood. Leukopenia may be induced by exposure to certain viruses or to radiation. It is often a side effect of various forms of cancer therapy, e.g., exposure to chemotherapeutic drugs, radiation and of infection or hemorrhage.

Hematopoietic progenitor cells also can be used in the treatment or prevention of neutropenia and, for example, in the treatment of such conditions as aplastic anemia, cyclic neutropenia, idiopathic neutropenia, Chediak-Higashi syndrome, systemic lupus erythematosus (SLE), leukemia, myelodysplastic syndrome, myelofibrosis, thrombocytopenia. Severe thrombocytopenia may result from genetic defects such as Fanconi's Anemia, Wiscott-Aldrich, or May-Hegglin syndromes and from chemotherapy and/or radiation therapy or cancer. Acquired thrombocytopenia may result from auto- or allo-antibodies as in Immune Thrombocytopenia Purpura, Systemic Lupus Erythromatosis, hemolytic anemia, or fetal maternal incompatibility. In addition, splenomegaly, disseminated intravascular coagulation, thrombotic thrombocytopenic purpura, infection or prosthetic heart valves may result in thrombocytopenia.

Thrombocytopenia may also result from marrow invasion by carcinoma, lymphoma, leukemia or fibrosis.

Many drugs may cause bone marrow suppression or hematopoietic deficiencies. Examples of such drugs are AZT, DDI, alkylating agents and anti metabolites used in chemotherapy, antibiotics such as chloramphenicol, penicillin, gancyclovir, daunomycin and sulfa drugs, phenothiazones, tranquilizers such as meprobamate, analgesics such as aminopyrine and dipyrone, anticonvulsants such as phenytoin or carbamazepine, antithyroids such as propylthiouracil and methimazole and diuretics. Transplantation of the hematopoietic progenitor cells can be used in preventing or treating the bone marrow suppression or hematopoietic deficiencies which often occur in subjects treated with these drugs.

Hematopoietic deficiencies may also occur as a result of viral, microbial or parasitic infections and as a result of treatment for renal disease or renal failure, e.g., dialysis. Transplantation of the hematopoietic progenitor cell populations may be useful in treating such hematopoietic deficiency.

Various immunodeficiencies, e.g., in T and/or B lymphocytes, or immune disorders, e.g., rheumatoid arthritis, may also be beneficially affected by treatment with the hematopoietic progenitor cells. Immunodeficiencies may be the result of viral infections (including but not limited to HIVI, HIVII, HTLVI, HTLVII, HTLVIII), severe exposure to radiation, cancer therapy or the result of other medical treatment.

In specific embodiments, the hematopoietic progenitor cells are used for the treatment of multiple myeloma, non-Hodgkin's lymphoma, Hodgkin's disease, neuroblastoma, germ cell tumors, autoimmune disorder (e.g., Systemic lupus erythematosus (SLE) or systemic sclerosis), amyloidosis, acute myeloid leukemia, acute lymphoblastic leukemia, chronic myeloid leukemia, chronic lymphocytic leukemia, myeloproliferative disorder, myelodysplastic syndrome, aplastic anemia, pure red cell aplasia, paroxysmal nocturnal hemoglobinuria, Fanconi anemia, Thalassemia major, Sickle cell anemia, Severe combined immunodeficiency (SCID), Wiskott-Aldrich syndrome, Hemophagocytic lymphohistiocytosis (HLH), or inborn errors of metabolism (e.g., mucopolysaccharidosis, Gaucher disease, metachromatic leukodystrophies or adrenoleukodystrophies). In some embodiments, the hematopoietic progenitor cells are used for the treatment of an inherited immunodeficient disease, an autoimmune disease and/or a hematopoietic disorder.

In one embodiment, the hematopoietic progenitor cells are for replenishment of hematopoietic cells in a patient who has undergone chemotherapy or radiation treatment. In a specific embodiment, the hematopoietic progenitor cells are administered to a patient that has undergone chemotherapy or radiation treatment. In a specific embodiment, the hematopoietic progenitor cells are administered to a patient who has HIV (e.g., for replenishment of hematopoietic cells in a patient who has HIV).

Preferred conditions treatable by the disclosed methods or cells include characterized by an inadequate amount or activity of immune cells. The immunodeficiency disorder may be primary or secondary. In one embodiment, the immunodeficiency disorder is a primary immunodeficiency disorder selected from: a T-cell, B-cell, or combined T-cell/B-cell immunodeficiency, such as severe combined immunodeficiency (SCID); an antibody deficiency, such as agammaglobulinemia; a complement deficiency, such as lupus; leukemia; lymphoma; an anemia, such as severe aplastic anemia; neutropenia; lymphopenia; or any condition associated with immune deficiency, such as Wiskott-Aldrich syndrome. In another embodiment, the immunodeficiency disorder is a secondary immunodeficiency disorder associated with an infectious disease including human immunodeficiency virus (HIV) or hepatitis. In another embodiment, the immunodeficiency disorder is a secondary immunodeficiency disorder associated with the administration of an immunosuppressive agent, such as fluorouracil, vincristine, cisplatin, oxoplatin, methotrexate, 3′-azido-3′-deoxythymidine, paclitaxel, doxetaxel, an anthracycline antibiotic, or mixtures thereof having a secondary immunosuppressive effect. All references, patents and patent applications disclosed herein are incorporated by reference with respect to the subject matter for which each is cited, which in some cases may encompass the entirety of the document.

As used herein, a “population” of cells refers to a group of at least 2 cells, e.g. 2 cells, 3 cells, 4 cells, 10 cells, 100 cells, 1000 cells, 10,000 cells, 100,000 cells or any value in between, or more cells. Optionally, a population of cells can be cells which have a common origin, e.g. they can be descended from the same parental cell, they can be clonal, they can be isolated from or descended from cells isolated from the same tissue, or they can be isolated from or descended from cells isolated from the same tissue sample. Preferably, the population of hematopoietic progenitor cells is substantially purified. As used herein, the term “substantially purified” means a population of cells substantially homogeneous for a particular marker or combination of markers. By substantially homogeneous is meant at least 80%, at least 85%, at least 90%, at least 95%, at least 96%, at least 97%, at least 98%, at least 99% or more homogeneous for a particular marker or combination of markers.

Unless clearly indicated to the contrary, the order of the steps or acts of the method of the invention is not necessarily limited to the order in which the steps or acts of the method are recited.

As used herein, each occurrence of terms such as “comprising” or “comprises” may optionally be substituted with “consisting of” or “consists of”.

The present disclosure is further illustrated by the following non-limiting examples.

### Examples

### Materials and Methods

### Human Embryonic Tissues

Human embryonic samples were obtained after elective medical termination of pregnancy. Each patient signed an informed consent to approve the research use of the samples. The study was approved by Ospedale San Raffaele Ethical Committee (TIGET-HPCT protocol). Samples were either used immediately as fresh tissues.

Human embryonic tissues (CS13) employed for ex vivo hematopoietic cultures were collected by human developmental biology resource (HDBR), Newcastle University, Newcastle, United Kingdom, with written informed consent and approval from the Newcastle and North Tyneside NHS Health Authority Joint Ethics Committee (08/H0906/21+5). The HDBR is regulated by the UK Human Tissue Authority (HTA; https://www.hta.gov.uk/) and operates in accordance with the relevant HTA Codes of Practice. The human embryonic tissues were dissociated for 50 minutes at 37° C. with 10 mg/ml Collagenase/Dispase (Sigma-Aldrich, 10269638001) in Phosphate Buffered Saline (PBS) with Ca²⁺ and Mg²⁺ (Sigma-Aldrich, D8662), supplemented with 7% heat-inactivated fetal bovine serum (FBS, Hyclone, 12389802), 1% Penicillin-Streptomycin (Lonza, DE17-603E) and 10 μg/ml DNAse I (Calbiochem, 260913) and filtered through a 40 μm cell strainer (Falcon, 352235), similarly to what was previously described (Ivanovs et al, 2011).

Human embryonic tissues (CS12-CS13) analyzed by RNA sequencing were incubated in medium containing 0.23% w/v collagenase Type I (Worthington Biochemical Corporation, NC9482366) for 30′ at 37° C. and filtered through a 70 μm cell strainer (70 μm, BD Biosciences).

### Human Pluripotent Stem Cells Maintenance and Differentiation

The use of human embryonic stem cells (hESC) was approved by the Ospedale San Raffaele Ethical Committee, included in the TIGET-HPCT protocol. The already established H1 (Thomson et al, 1998) and RUNX1C-EGFP H9 (Ng et al, 2016a) hESCs lines as well as corrected SCID-X1 iPSCs were grown on irradiated MEF feeders in hES medium defined as DMEM/F12 medium (Corning, L022046-10092CVR) supplemented with 25% of KnockOut™ Serum Replacement (Thermo Fisher Scientific, 10828028), 1% Penicillin-Streptomycin (Lonza, DE17-603E) 2 mM L-Glutamine (Lonza, BE17-605E), 0.1% p-Mercaptoethanol (Sigma Aldrich, M3148), 0.7% of MEM Non-Essential Amino Acids Solution (Thermo Fisher Scientific, 11140035). 1 μg/ml Ciprofloxacin HCl (Sigma-Aldrich, PHR1044-1G) and 20-30 ng/ml human recombinant basic fibroblast growth factor (bFGF, R&D, 233-FB-500/CF) were added to hES medium right before usage. Cells were maintained and expanded at 37° C., 21% 02, 5% CO2.

For differentiation, hPSC were cultured on Matrigel-coated plasticware (Corning Life Sciences, 356230) for 24 hours, followed by embryoid body (EB) generation. Embryoid body (EB) aggregates were resuspended in SFD media define as 75% IMDM (Corning, 15343531) 25% Ham's F12 (Corning, 10-080-CVR), 0.005% BSA—Fraction V, B27 supplement (Thermo Fisher Scientific Cat #12587010), N2 supplement (Thermo Flsher Scientific Cat #17502048), 1% Penicillin-Streptomycin, 1 μg/ml Ciprofloxacin HCl. The differentiation media was supplemented as previously described (Ditadi & Sturgeon, 2016; Ditadi et al, 2015). Briefly, the first day of differentiation SFD medium was supplemented with 2 mM L-glutamine, 1 mM ascorbic acid (Sigma Aldrich, A4544), 400 μM 1-Thioglycerol solution (Sigma-Aldrich, M6145), 150 μg/ml transferrin (R&D, 2914-HT), and 10 ng/ml BMP4 (R&D, 314-BP-MTO). 24 hours later, 5 ng/ml bFGF (R&D, 233-FB-500/CF) was added. On the second day of differentiation, either 3 μM CHIR99021 (Cayman Chemical Company, CT99201), or 3 μM IWP2 (Cayman Chemical Company, 004CA13951) were added, as indicated. On the third day, embryoid bodies (EBs) were changed to StemPro-34 media (Thermo Fisher Scientific, 10639011) supplemented with Penicillin-Streptomycin, L-glutamine, ascorbic acid, MTG and transferrin, as above, with additional 5 ng/ml bFGF and 15 ng/ml VEGF (R&D, MAB3572). On day 6, 10 ng/ml IL-6 (130-093-934), 25 ng/ml IGF-1 (130-093-887), 5 ng/ml IL-11 (130-103-439), 50 ng/ml SCF (130-096-696), 2 U/ml EPO (Peprotech, 100-64). All cytokines were purchased from Miltenyi Biotec, unless indicated differently. All differentiation cultures were maintained at 37° C. All embryoid bodies and mesodermal aggregates were cultured in a 5% CO₂, 5% O₂, 90% N₂.

### NK- and T-Lineage Differentiation

OP9 cells expressing murine DLL1 or human DLL4 were described previously (Schmitt & Zúñiga-Pflücker, 2002; Mohtashami et al, 2010). To test the NK- or the T-cell potential, 100-5000 candidate cells isolated by FAC-sorting as indicated were seeded on OP9DLL1 or OP9DLL4-coated 24-well plates, respectively. For both NK- and T-cell differentiations, the cells were cultured in Alpha MEM (Thermo Fisher, 12000063) supplemented with 2.2 g/L sodium bicarbonate (Corning, 61-065-RO), 20% FBS (HyClone), 1% Penicillin-Streptomycin, 2 mM glutamine (Thermo Flsher Scientific) and 400 μM 1-Thioglycerol solution supplemented with lineage specific cytokines. For NK-specific differentiation, cultures were supplemented with 5 ng/ml IL7 (130-095-362), 5 ng/ml FLT3L (130-096-479), 10 ng/ml IL15 (130-95-765) and for the first 7 days of differentiation 30 ng/ml IL3 (130-095-070). All cytokines were purchased from Miltenyi Biotec. The cells maintained for 14 days on the same stromal cells. Every 7 days, the culture was supplemented with fresh medium. The differentiation was prolonged to 21 days for LDA assays in which 1, 5, 10, 25, 30, 50 or 100 cells were directly seeded on OP9DLL1-coated 96-well during FAC-Sorting. NK-lymphoid output was assayed by FACS analysis. Frequency was calculated by extreme limiting dilution analysis (ELDA) (http://bioinf.wehi.edu.au/software/elda/) (Hu & Smyth, 2009). For T-lineage differentiation, cultures were supplemented 5 ng/ml IL7, 5 ng/ml FLT3L and for the first 5 days of differentiation 50 ng/ml SCF. The cells were split every 4-5 days by vigorous pipetting and passaging through a 40 μm cell strainer and plated on freshly seeded OP9DLL4. T-lymphoid output was assayed by FACS analysis after 21-24 days of differentiation. Cells were analyzed using a LSR Canto II flow cytometer (BD).

### CFC Generation Assay

The CFC generation assay was performed as previously described (Kennedy et al, 2012). Briefly, sorted cells were cultured on irradiated OP9DLL1 monolayers in Alpha-MEM (Alpha-MEM, ThermoFisher, 12000063) supplemented with 20% FBS (HyClone), 1% Penicillin-Streptomycin 2 mM L-glutamine, 30 ng/ml TPO (Miltenyi Biotec, 130-095-747), 10 ng/ml BMP4, 50 ng/ml, 25 ng/ml IGF1, 10 ng/ml IL11, 10 ng/ml FLT3L, 4 U/ml EPO. After five days, cells were collected using 0.25% Trypsin-EDTA (Thermo Fisher Scientific, 25-200-056) for 3′ at 37° C. Cells were filtered then filtered through a 40 μM filter and seeded on methylcellulose medium (Stemcell Technologies, H4034). WNTi hematopoietic cultures were seeded on methylcellulose supplemented with 150 ug/ml transferrin, 50 ng/ml TPO, 10 ng/ml VEGF, 50 ng/ml IL3, SCF 100 ng/ml, 10 ng/ml IL6, 50 ng/ml IGF1, 5 ng/ml IL11, 4 U/ml EPO, GM-CSF 1 ng/ml (Miltenyi Biotec, 130-093-864), 6 uM SB 431542 (Cayman Chemical Company, 13031). WNTd hematopoietic cultures were seeded on methylcellulose supplemented with 150 ug/ml transferrin, 50 ng/ml TPO, 10 ng/ml VEGF, 10 ng/ml IL6, 50 ng/ml IGF1, 5 ng/ml IL11, 4 U/ml EPO. Colonies' number and morphology was evaluated after fifteen days by light microscopy.

### Endothelial to Hematopoietic (EHT) Assay

CD43^{neg}CD184^{neg}CD73^{neg}DLL4^{neg} were isolated at day 8 of WNTd hematopoietic culture and reaggregated overnight at 3×10⁵ cells/ml as previously described (Ditadi et al, 2015). The cells were seeded in Stempro media (Life Technologies, 10639011), complemented with 1% glutamine, 50 μg/ml ascorbic acid, 150 μg/ml transferrin, 400 μM 1-Thioglycerol solution, 30 ng/ml TPO, 10 ng/ml VEGF, 5 ng/ml bFGF, 30 ng/ml IL3, 100 ng/ml SCF, 25 ng/ml IGF1, 10 ng/ml IL6, 10 ng/ml BMP4, 5 ng/ml IL11, 2 U/ml EPO. Aggregates were then transferred onto thin-layer Matrigel-coated plasticware where they were cultured for an additional 3 days in the same media. Single CD34⁺CD43^{neg}CD184^{neg}CD73^{neg}DLL4^{neg}CD32+/CD44⁺ cells were FAC-sorted directly onto a Matrigel-coated well of 96-well plate at day 8 of WNTd hematopoietic cultures. Cells were cultured as above. Hematopoietic and non-hematopoietic clones were evaluated by light microscopy and FACS analysis after 10-14 days of culture.

### Cell Staining, Flow Cytometry and Cell Sorting

Samples for FACS analysis or cell sorting were incubated with antibody mixes for 15-30′ at 4° C. The antibodies employed are listed in the table below. Dead cells were excluded using 7AAD during staining. Cells were sorted with FACSAria II (BD). Sorting gates were set using appropriate fluorescence minus one (FMO) and single staining controls. FACS-analysis were performed either at FACS Canto, (BD Biosciences) or Cytoflex S (Beckman Coulter).

### Antibodies List

Anti-human antibodies

| | | | | |
|---|---|---|---|---|
| ACE (BB9) | — | BD | 557813 | 2:100 |
| CD184 | BV421 | BD | 562448 | 1:100 |
| CD32 | PE | BD | 303206 | 2:100 |
| CD34 | APC | Beckman Coulter | IM2472 | 2:100 |
| CD34 | PE Cyanine 7 | Biolegend | 343616 | 1:400 |
| CD34 | PE Cyanine 7 | eBioscience | 25034942 | 1:400 |
| CD4 | PE Cy7 | BD | 560649 | 2:100 |
| CD43 | FITC | BD | 555475 | 10:100 |
| CD44 | PE | Miltenyi | 130-113-342 | 2:100 |
| CD45 | APC Cyanine 7 | Biolegend | 368516 | 1:100 |
| CD45 | FITC | Beckman Coulter | A07782 | 2:100 |
| CD45 | BV421 | BioLegend | 304032 | 2:100 |
| CD5 | PE | BioLegend | 300607 | 5:100 |
| CD56 | BV421 | BD | 740076 | 3:100 |
| CD7 | APC | BD | 561604 | 2:100 |
| CD73 | BV421 | BD | 562430 | 3:100 |
| CD8 | PE | Biolegend | 31051 | 2:100 |
| DLL4 | APC | Biolegend | 346508 | 1:100 |
| anti-IgG1 | PE | Southern | 1070-05 | 1:1000 |
| | | Biotechnology | | |
| Fcr Blocking reagent | — | Miltenyi | 130-059-901 | 20:100 |
| human | | | | |
| | 7AAD | BD | 559925 | 1:100 |

### Results

To overcome the above limitations and develop broadly applicable strategies for the enrichment of H ECs, inventors used transcriptomic analysis of endothelial populations of the human embryo known to display hematopoietic potential for the identification of cell-surface markers specific for human HECs. Here inventors report that the Fc receptor 0032 is expressed on human embryonic endothelial cells with hemogenic potential and identifies hPSC-derived HECs with higher specificity than other reported HEC markers. To validate 0032 as a marker of HECs, inventors FAC-sorted the different 0032 fractions from CD34⁺CD43^{neg}CD45^{neg} endothelial cells of AGM region and YS dissected from two CS13 embryos (E5, E6) and assessed their hematopoietic potential in vitro (FIG. 1A, B, FIG. 4A). While at the population level, both CD32+ and CD32^{neg} fractions, regardless of their localization, showed NK- and T-lymphoid potential (FIGS. 4B,C), the CD32+ fraction from both the YS and AGM region generated more clonogenic progenitors with erythro-myeloid potential (FIGS. 1C,D). Altogether, these results support the hypothesis that CD32 can identify a subset of endothelial cells with robust hematopoietic potential in the human embryo.

CD32 Defines HECs with Multilineage Potential in hPSC Differentiating Cultures.

Inventors next investigated whether CD32 can be a reliable marker for isolating HECs from hPSC differentiating cultures. Inventors monitored its expression in HECs derived from both IWP2-treated WNT-independent (WNTi) cultures yielding extra-embryonic-like hematopoietic progenitors and WNTd intra-embryonic-like definitive HECs (Sturgeon et al, 2014; Ditadi et al, 2015). Contrary to ACE, CD32 identifies a subset of CD34⁺ cells, including CD34⁺CD43^{neg}CD73^{neg}CD184^{neg}DLL4^{neg} cells that contain HECs with multipotent hematopoietic potential (FIGS. 2A,B; FIG. 5A) (Ditadi et al, 2015). Notably, using a H9 hESC reporter line for RUNX1C expression (Ng et al, 2016b), inventors confirmed that CD32 expression demarcates endothelial cells that are not already undergoing active EHT (FIGS. 2B,C). Inventors therefore assessed the hematopoietic potential of different CD32/DLL4 fractions within the CD34⁺CD43^{neg}CD73^{neg}CD184^{neg} cell population (FIG. 5D). In both WNTi and WNTd cultures, inventors confirmed that the hematopoietic potential segregated almost exclusively to DLL4^{neg} cells, with the CD32+DLL4^{neg} cells generating significantly more erythro-myeloid clonogenic progenitors in both H1 and H9 hESC lines (FIG. 2C, FIGS. 5E,F). Inventors then assessed the lymphoid potential in both WNTi and WNTd cultures, analyzing the defining lymphoid lineage for each program, i.e., NK- and T-cells, respectively (Dege et al, 2020; Kennedy et al, 2012). While within the WNTi DLL4^{neg} cell population, both CD32 fractions possessed NK-cell potential, in some, but not all, experiments, CD32+DLL4^{neg} cells yielded a higher proportion of NK cells (FIGS. 5G,H). This prompted us to determine the frequency of NK-progenitors in the CD32 fractions with limiting dilution analysis (LDA), which revealed a 16-fold enrichment in CD32+ HECs (1:15 vs 1:244; FIG. 2D, FIG. 51). In WNTd cultures, the segregation of T-cell potential with CD32 expression was more evident, as only CD32+DLL4^{neg} cells could robustly generate CD4⁺CD8⁺ T-cells when 350 or fewer cells were assayed (FIGS. 2E,F). Collectively, these results show that CD32 expression in CD34⁺CD43^{neg}CD73^{neg}CD184^{neg}DLL4^{neg} cells demarcates a subpopulation endowed with robust multilineage hematopoietic potential across different hematopoietic developmental programs in hPSC cultures.

### CD32 is a Highly Specific HEC Marker.

Inventors next wanted to compare the specificity of CD32 as hPSC-derived HEC marker against other surface proteins used to define HECs, in particular CD44 (Zeng et al, 2019; Oatley et al, 2020). Similarly to ACE, CD44 marks most of day 8 WNTd CD34⁺ cells, in line with the reported CD44 expression in both arterial ECs and HECs (FIGS. 3A,B) (Robert-Moreno et al, 2008; Zeng et al, 2019; Oatley et al, 2020). Inventors confirmed that, within CD34⁺CD43^{neg}CD73^{neg}CD184^{neg}DLL4^{neg} cells, the CD44⁺ fraction is enriched for HECs as this fraction generates significantly more clonogenic progenitors (FIG. 3C, FIG. 6A). As such, to compare the specificity of CD32 and CD44, inventors turned to the single-cell EHT assay inventors previously described (Ditadi et al, 2015). This clonal analysis revealed that the CD32+ subfraction was highly enriched for HECs as 89.2% of the cells that formed a clone in the EHT assay (112 out of 576) generated only CD45⁺ hematopoietic cells (FIG. 3D, FIGS. 6B,C). In contrast, the CD44⁺ fraction contains equal proportions of progenitors with hematopoietic and non-hematopoietic potential (FIG. 3D, FIG. 6D). This single cell analysis indicates that CD32 is a reliable marker for hPSC-derived HECs, whose specificity is superior to the one of CD44, often used to identify human HECs. Inventors' identification of CD32 as a HEC-specific marker now enables easy and routine access to highly enriched populations of blood progenitors from a broad range of hPSC lines, including those that do not differentiate efficiently to hematopoietic lineages using current protocols. For example, inventors generated corrected SCID-X1 induced pluripotent stem cell (iPSC) lines whose differentiation according to standard protocols yields CD34⁺ cells containing very few hematopoietic progenitors. Using this line, while the isolation of 5000 CD34⁺CD43^{neg} cells failed to yield hematopoietic cells, 100 CD34⁺CD43^{neg}CD73^{neg}CD184^{neg}DLL4^{neg}CD32+ cells robustly generate CD45⁺CD56⁺ NK-cells in vitro (FIGS. 3E,F).

### DISCUSSION

The precise identification of human HECs will allow to characterize this transient population and identify what regulates their transition to blood as well as to determine their identity, which is still subject to debate. The present inventors, using transcriptomic analysis of hemogenic populations found in the human embryo, identified CD32 as a marker whose expression is upregulated in HECs. CD32 expression can be used in combination with other endothelial markers to precisely isolate HECs from both the human embryo and hPSC differentiating cultures across different hematopoietic programs. In particular, CD32 expression is more specific for hPSC-derived HECs than CD44, another marker known to be expressed in HECs as well as in arterial cells (Robert-Moreno et al, 2008; Zeng et al, 2019; Oatley et al, 2020).

The expression of Fc receptors, like CD32, was already observed in association with the endothelial marker VE-cadherin in hematopoietic progenitors within the murine YS (McGrath et al, 2015). In addition, CD32 is a marker of other specialized endothelia, as it is found on LSECs and can be expressed in lymphatic endothelial cells (LECs) (Strauss et al, 2017; Gage et al, 2020; Xiang et al, 2020). Another marker classically associated with lymphatic vessels, lymphatic vessel endothelial hyaluronan receptor-1 (LYVE1) is expressed by LSECs, LECs as well as the YS and umbilical and vitelline endothelia, including cells displaying hematopoietic potential (Lee et al, 2016). This similarity between the HECs and LECs raises the interesting possibility of common mechanisms regulating the emergence of these lineages. Notably, both lineages develop in close association with vascular endothelial cells (major arteries for HECs, major veins for LECs) (DeBruijn et al, 2000; Hogan & Schulte-Merker, 2017) and their specification involve considerable remodeling and upregulation of ribosomal biogenesis (Zeng et al, 2019; Fadlullah et al, 2021; Koltowska et al, 2021). A common origin for HECs and LECs lineages could also be possible (Stanczuk et al, 2015). The prospective isolation of HECs and LEC endothelial progenitors, before their respective transition to blood and lymphatic fates, will allow to interrogate their lineage relationship and identify potential common mediators of their development. Despite the well-described heterogeneity of HECs, our findings indicate that CD32 is expressed in HECs harboring multilineage hematopoietic potential isolated from different anatomic locations of the human embryo and in HECs derived from hPSC differentiations recapitulating distinct hematopoietic programs. While these data suggest that CD32 is a pan-HECs marker, inventors could not test whether CD32 is expressed on HSC-competent HECs, since culture conditions supporting human HSC specification from HECs, even from human embryonic explant cultures, have not been identified (Easterbrook et al, 2019). Notably, CD32+ hemogenic cells do not display DLL4 expression, despite their robust lymphoid potential. Further experiments are needed to investigate whether this population acquires an arterial signature, including DLL4 and CXCR4 expression, before undergoing EHT, similarly to what described by others for HECs in vitro and in vivo (Dignum et al, 2021; Uenishi et al, 2018; Zeng et al, 2019). Nonetheless, inventors believe that the precise isolation of HECs using CD32 will allow to molecularly resolve HEC heterogeneity and determine what regulates the different potential displayed by spatio-temporal distinct HECs. The specific expression pattern of CD32 in human embryonic and hPSC-derived HECs, suggest that this receptor plays some functional role in the human hematopoietic lineage, perhaps as early as the hematopoietic specification during development. The fact that in immunodeficient humans, HECs can undergo EHT to generate functional blood cells in the absence of circulating antibodies, the best characterized ligands of CD32, suggests that CD32 could potentially functionally regulate blood development via the of binding alternative ligands, such as the acute phase reactant C-reactive protein or the multifunctional protein fibrinogen-like protein 2 (FGL2) (Liu et al, 2008; Sundgren et al, 2011). In particular, the latter is intriguing since the Fg/2-null mouse model shows developmental defects and high rate of early embryonic lethality before E9.5 (Clark et al, 2004).

In summary, the present data demonstrate that expression of CD32 marks HECs and that it can be used to highly enrich populations of hematopoietic precursors from a broad range of hPSC lines, including those for which current differentiation protocols into hematopoietic lineages are not optimal. The present findings will allow a deeper understanding of this central element of hematopoietic development, which will translate into optimized protocols to generate therapeutic blood products from hPSCs.

### CD32 Identifies HECs in a Specific Notch-Independent State.

In the attempt to further characterize hPSC-derived CD32+ cells, inventors next asked if these cells have transcriptional similarity to HECs found in the developing human embryo. We analyzed the transcriptomic profile of the CD32+ fraction sorted from day 8 WNTd hematopoietic cultures and, since HECs in the DA are found in close contact with non-hemogenic cells arterial cells (DeBruijn et al, 2000), we included CD34⁺CD43^{neg}CD184⁺CD73⁺DLL4⁺ cells (referred to as DLL4⁺) as control sample (Ditadi et al, 2015). While DLL4⁺ cells displayed a significant enrichment for genes whose expression is associated with arterial fate in vivo (e.g., CXCR4, DLL4, HEY1, HEY2, SOX17, GJA5) (Calvanese et al, 2022), CD32+ cells were enriched for the expression of genes associated with HECs and their hematopoietic progression in vivo, including RUNX1, GF/1, MYCN and RAB27B (FIG. 7A)(Calvanese et al, 2022). We then wondered whether FCGR2B expression could further refine the identification of HECs using available human CS14-15 AGM single cell RNA sequencing (scRNA-seq) data (Calvanese et al, 2022). In this dataset, while CDH5⁺RUNX1⁺PTPRC^{neg}FCGR2B^{neg} HECs display an enrichment for genes associated with arterial endothelium, the expression of genes characteristic of hematopoietic commitment segregates within CDH5⁺RUNX1⁺PTPRC^{neg}FCGR2B⁺ expressing cells (FIG. 7B).

These data suggest that transcriptionally distinct states of HECs can be identified. To assess HEC heterogeneity and interrogate whether FCGR2B expression could define a specific state of HECs, we performed single-cell RNAseq (scRNAseq) of day 8 WNTd CD34⁺CD43^{neg}CD184^{neg}CD73^{neg} cells as they contain HECs as well as other endothelial progenitors and HEC precursors (Ditadi et al, 2015). Unsupervised clustering revealed 22 transcriptionally distinct clusters which were mostly annotated to the major endothelial cell fates (FIGS. 7C,D). This scRNAseq analysis confirmed that WNTd HECs showed transcriptional heterogeneity as a total of 6 clusters were enriched for cells differentially expressing RUNX1 (FIGS. 7C,E), including one with enriched expression of FCGR2B (cluster 11 in FIGS. 7C,F). Pseudotime analysis by Monocle3 revealed that FCGR2B+ HECs represent an intermediate state for the progression of RUNX1⁺KCNK17⁺H19⁺FCGR2B^{neg} HECs (Zhou et al, 2019) to RUNX1⁺ cells expressing hematopoietic genes such as SPN (despite being CD43^{neg}) and MYB (FIG. 8A-C). We next performed GSEA and ORA across this progression to dissect the unique features of FCGR2B⁺ HECs and identify state-specific gene expression profiles proper of HECs with distinct characteristics. In particular, we observed that the gradual loss of endothelial identity of H19⁺HECs begins with the downregulation of genes associated with extracellular organization, cell adhesion and cytoskeletal remodeling (FIG. 8D). The progression to the FCGR2B⁺ HEC cluster is associated with an enrichment of the expression of several ribosomal protein genes, consistently with the role of RUNX1 in regulating ribosome biogenesis (Cai et al, 2015), a key process for the emergence of blood cells (FIG. 8D). Next, concomitantly to the upregulation of hematopoietic genes, HECs also display increased expression of genes associated with cell motility as well as cell cycle progression (FIG. 8A). Indeed, using scRNA-seq data to infer the cell cycle state across HEC states, we observed that during the progression from H19⁺ to FCGR2B⁺ cluster (i.e., from clusters 0, 1, 2 to cluster 11), cells are mostly in G1 phase, while cells in the MYB⁺ HEC clusters (i.e., clusters 16, 17) are mostly in S/G2/M phase (FIG. 8E).

Interestingly, the MYB⁺ HEC clusters (i.e., clusters 16, 17) negatively correlated with expression of genes associated to the activation of Notch signaling (FIG. 7c). Since Notch signaling is an essential driver of stage-specific intra-embryonic emergence of hematopoietic cells (Souilhol et al, 2016), we further analyzed the expression trend according to pseudotime of the well characterized NOTCH target genes in the DA, i.e., HES1, HEY1 and HEY2 (FIG. 8C). The analysis revealed that HES1 expression peaks in cells belonging to cluster 11, marked by FCGR2B differential expression, while HEY1 and HEY2 expression peaks in cells that precede FCGR2B⁺ cells in this pseudotime (FIG. 8C). This suggests that FCGR2B expression might identify a Notch-independent state of HECs. To test this hypothesis, we added the chemical γ-secretase inhibitor L-685,458 (γSi) to the HEC culture of day 8 WNTd CD32+ as well as CD32^{neg} cells, as some of the latter will generate CD32+ cells. While CD32^{neg} cells gave rise to hematopoietic progenitors in a Notch-dependent manner, the chemical inhibition of Notch signaling did not impair the generation of CD45⁺ hematopoietic progenitors from CD32+ cells (FIGS. 8F,G). Altogether these results show that CD32 expression defines the temporal Notch-requirement within the HECs and that HECs activate a cell division program to give rise to hematopoietic progeny.

### REFERENCES

Bertrand J Y, Chi N C, Santoso B, Teng S, Stainier D Y R & Traver D (2010) Haematopoietic stem cells derive directly from aortic endothelium during development. Nature 464: 108
Boisset J-C, Cappellen W van, Andrieu-Soler C, Galjart N, Dzierzak E & Robin C (2010) In vivo imaging of haematopoietic cells emerging from the mouse aortic endothelium. Nature 464: 116-120
Cai X, Gao L, Teng L, Ge J, Oo Z M, Kumar A R, Gilliland D G, Mason P J, Tan K & Speck N A (2015) Runx1 Deficiency Decreases Ribosome Biogenesis and Confers Stress Resistance to Hematopoietic Stem and Progenitor Cells. Cell Stem Cell 17: 165-177
Calvanese V, Capellera-Garcia S, Ma F, Fares I, Liebscher S, Ng E S, Ekstrand S, Aguadé-Gorgorió J, Vavilina A, Lefaudeux D, et al (2022) Mapping human haematopoietic stem cells from haemogenic endothelium to birth. Nature: 1-7
Clark D A, Foerster K, Fung L, He W, Lee L, Mendicino M, Markert U R, Gorczynski R M, Marsden P A & Levy G A (2004) The fgl2 prothrombinase/fibroleukin gene is required for lipopolysaccharide-triggered abortions and for normal mouse reproduction. Mhr Basic Sci Reproductive Medicine 10: 99-108
DeBruijn M F T R, Speck N A, Peeters M C E & Dzierzak E (2000) Definitive hematopoietic stem cells first develop within the major arterial regions of the mouse embryo. Embo J 19: 2465-2474
Dege C, Fegan K H, Creamer J P, Berrien-Elliott M M, Luff S A, Kim D, Wagner J A, Kingsley P D, McGrath K E, Fehniger T A, et al (2020) Potently Cytotoxic Natural Killer Cells Initially Emerge from Erythro-Myeloid Progenitors during Mammalian Development. Dev Cell 53: 229-239.e7
Dignum T, Vamum-Finney B, Srivatsan S R, Dozono S, Waltner O, Heck A M, Ishida T, Nourigat-McKay C, Jackson D L, Rafii S, et al (2021) Multipotent progenitors and hematopoietic stem cells arise independently from hemogenic endothelium in the mouse embryo. Cell Reports 36: 109675
Ditadi A & Sturgeon C M (2016) Directed differentiation of definitive hemogenic endothelium and hematopoietic progenitors from human pluripotent stem cells. Methods 101: 65-72
Ditadi A, Sturgeon C M, Tober J, Awong G, Kennedy M, Yzaguirre A D, Azzola L, Ng E S, Stanley E G, French D L, et al (2015) Human definitive haemogenic endothelium and arterial vascular endothelium represent distinct lineages. Nat Cell Biol 17: 580-591
Dzierzak E & Bigas A (2018) Blood Development: Hematopoietic Stem Cell Dependence and Independence. Cell Stem Cell 22: 639-651
Easterbrook J, Rybtsov S, Gordon-Keylock S, Ivanovs A, Taoudi S, Anderson R A & Medvinsky A (2019) Analysis of the Spatiotemporal Development of Hematopoietic Stem and Progenitor Cells in the Early Human Embryo. Stem Cell Reports
Fadlullah M Z, Neo W H, Lie-a-ling M, Thambyrajah R, Patel R, Mevel R, Aksoy I, Khoa N D, Savatier P, Fontenille L, et al (2021) Murine AGM single-cell profiling identifies a continuum of hemogenic endothelium differentiation marked by ACE. Blood
Frame J M, Fegan K H, Conway S J, McGrath K E & Palis J (2016) Definitive Hematopoiesis in the Yolk Sac Emerges from Wnt-Responsive Hemogenic Endothelium Independently of Circulation and Arterial Identity. Stem Cells 34: 431-444
Gage B K, Liu J C, Innes B T, MacParland S A, McGilvray I D, Bader G D & Keller G M (2020) Generation of Functional Liver Sinusoidal Endothelial Cells from Human Pluripotent Stem-Cell-Derived Venous Angioblasts. Cell Stem Cell 27: 254-269.e9
Hogan B M & Schulte-Merker S (2017) How to Plumb a Pisces: Understanding Vascular Development and Disease Using Zebrafish Embryos. Dev Cell 42: 567-583
Hu Y & Smyth G K (2009) ELDA: Extreme limiting dilution analysis for comparing depleted and enriched populations in stem cell and other assays. J Immunol Methods 347: 70-78
Ivanovs A, Rybtsov S, Welch L, Anderson R A, Turner M L & Medvinsky A (2011) Highly potent human hematopoietic stem cells first emerge in the intraembryonic aorta-gonad-mesonephros region. J Exp Medicine 208: 2417-2427
Jaffredo T, Gautier R, Eichmann A & Dieterlen-Libvre F (1998) Intraaortic hemopoietic cells are derived from endothelial cells during ontogeny. Dev Camb Engl 125: 4575-83
Kennedy M, Awong G, Sturgeon C M, Ditadi A, LaMotte-Mohs R, Zúñiga-Pflücker J C & Keller G (2012) T Lymphocyte Potential Marks the Emergence of Definitive Hematopoietic Progenitors in Human Pluripotent Stem Cell Differentiation Cultures. Cell Reports 2: 1722-1735
Kissa K & Herbomel P (2010) Blood stem cells emerge from aortic endothelium by a novel type of cell transition. Nature 464: 112
Koltowska K, Okuda K S, Gloger M, Rondon-Galeano M, Mason E, Xuan J, Dudczig S, Chen H, Arnold H, Skoczylas R, et al (2021) The RNA helicase Ddx21 controls Vegfc-driven developmental lymphangiogenesis by balancing endothelial cell ribosome biogenesis and p53 function. Nat Cell Biol 23: 1136-1147
Lancrin C, Sroczynska P, Stephenson C, Allen T, Kouskoff V & Lacaud G (2009) The haemangioblast generates haematopoietic cells through a haemogenic endothelium stage. Nature 457: 892
Lee L K, Ghorbanian Y, Wang W, Wang Y, Kim Y J, Weissman I L, Inlay M A & Mikkola H K A (2016) LYVE1 Marks the Divergence of Yolk Sac Definitive Hemogenic Endothelium from the Primitive Erythroid Lineage. Cell Reports 17: 2286-2298
Liu H, Shalev I, Manuel J, He W, Leung E, Crookshank J, Liu M F, Diao J, Cattral M, Clark D A, et al (2008) The FGL2-FcγRIIB pathway: A novel mechanism leading to immunosuppression. Eur J Immunol 38: 3114-3126
McGrath K E, Frame J M, Fegan K H, Bowen J R, Conway S J, Catherman S C, Kingsley P D, Koniski A D & Palis J (2015) Distinct Sources of Hematopoietic Progenitors Emerge before HSCs and Provide Functional Blood Cells in the Mammalian Embryo. Cell Reports 11: 1892-1904
Mohtashami M, Shah D K, Nakase H, Kianizad K, Petrie H T & Zúñiga-Pflücker J C (2010) Direct Comparison of Dl11- and D114-Mediated Notch Activation Levels Shows Differential Lymphomyeloid Lineage Commitment Outcomes. J Immunol 185: 867-876
Ng E S, Azzola L, Bruveris F F, Calvanese V, Phipson B, Vlahos K, Hirst C, Jokubaitis V J, Yu Q C, Maksimovic J, et al (2016a) Differentiation of human embryonic stem cells to HOXA+ hemogenic vasculature that resembles the aorta-gonad-mesonephros. Nat Biotechnol 34: 1168-1179
Ng E S, Azzola L, Bruveris F F, Calvanese V, Phipson B, Vlahos K, Hirst C, Jokubaitis V J, Yu Q C, Maksimovic J, et al (2016b) Differentiation of human embryonic stem cells to HOXA+ hemogenic vasculature that resembles the aorta-gonad-mesonephros. Nat Biotechnol 34: 1168-1179
North T, Gu T L, Stacy T, Wang Q, Howard L, Binder M, Marin-Padilla M & Speck N A (1999) Cbfa2 is required for the formation of intra-aortic hematopoietic clusters. Dev Camb Engl 126: 2563-75
Oatley M, Bölükbasi O V, Svensson V, Shvartsman M, Ganter K, Zirngibl K, Pavlovich P V, Milchevskaya V, Foteva V, Natarajan K N, et al (2020) Single-cell transcriptomics identifies CD44 as a marker and regulator of endothelial to haematopoietic transition. Nat Commun 11: 586
Orkin S H & Zon L I (2008) Hematopoiesis: An Evolving Paradigm for Stem Cell Biology. Cell 132: 631-644
Robert-Moreno A, Guiu J, Ruiz-Herguido C, López M E, Inglés-Esteve J, Riera L, Tipping A, Enver T, Dzierzak E, Gridley T, et al (2008) Impaired embryonic haematopoiesis yet normal arterial development in the absence of the Notch ligand Jagged1. Embo J 27: 1886-1895
Schmitt T M & Zúñiga-Pflücker J C (2002) Induction of T Cell Development from Hematopoietic Progenitor Cells by Delta-like-1 In Vitro. Immunity 17: 749-756
Souilhol C, Lendinez J G, Rybtsov S, Murphy F, Wilson H, Hills D, Batsivari A, Binagui-Casas A, McGarvey A C, MacDonald H R, et al (2016) Developing HSCs become Notch independent by the end of maturation in the AGM region. Blood 128: 1567-1577
Stanczuk L, Martinez-Corral I, Ulvmar M H, Zhang Y, Lavifia B, Fruttiger M, Adams R H, Saur D, Betsholtz C, Ortega S, et al (2015) cKit Lineage Hemogenic Endothelium-Derived Cells Contribute to Mesenteric Lymphatic Vessels. Cell Reports 10: 1708-1721
Stefanska M, Batta K, Patel R, Florkowska M, Kouskoff V & Lacaud G (2017) Primitive erythrocytes are generated from hemogenic endothelial cells. Scientific Reports 7: 6401
Strauss O, Phillips A, Ruggiero K, Bartlett A & Dunbar P R (2017) Immunofluorescence identifies distinct subsets of endothelial cells in the human liver. Sci Rep-uk 7: 44356
Sturgeon C M, Ditadi A, Awong G, Kennedy M & Keller G (2014) Wnt signaling controls the specification of definitive and primitive hematopoiesis from human pluripotent stem cells. Nat Biotechnol 32: 554-561
Sundgren N C, Zhu W, Yuhanna I S, Chambliss K L, Ahmed M, Tanigaki K, Umetani M, Mineo C & Shaul P W (2011) Coupling of Fc&ggr; Receptor I to Fc&ggr; Receptor IIB by Src Kinase Mediates C-Reactive Protein Impairment of Endothelial Function. Circ Res 109: 1132-1140
Swiers G, Baumann C, O'Rourke J, Giannoulatou E, Taylor S, Joshi A, Moignard V, Pina C, Bee T, Kokkaliaris K D, et al (2013a) Early dynamic fate changes in haemogenic endothelium characterized at the single-cell level. Nat Commun 4: 2924
Swiers G, Rode C, Azzoni E & Bruijn MFTR de (2013b) A short history of hemogenic endothelium. Blood Cells, Molecules, and Diseases 51: 206-212
Thambyrajah R, Mazan M, Patel R, Moignard V, Stefanska M, Marinopoulou E, Li Y, Lancrin C, Clapes T, Möröy T, et al (2016) GFI1 proteins orchestrate the emergence of haematopoietic stem cells through recruitment of LSD1. Nature Cell Biology 18: 21-32
Thomson J A, Itskovitz-Eldor J, Shapiro S S, Waknitz M A, Swiergiel J J, Marshall V S & Jones J M (1998) Embryonic Stem Cell Lines Derived from Human Blastocysts. Science 282: 1145-1147
Uenishi G I, Jung H S, Kumar A, Park M A, Hadland B K, McLeod E, Raymond M, Moskvin O, Zimmerman C E, Theisen D J, et al (2018) NOTCH signaling specifies arterial-type definitive hemogenic endothelium from human pluripotent stem cells. Nature Communications 9: 1828
Xiang M, Grosso R A, Takeda A, Pan J, Bekkhus T, Brulois K, Dermadi D, Nordling S, Vanlandewijck M, Jalkanen S, et al (2020) A Single-Cell Transcriptional Roadmap of the Mouse and Human Lymph Node Lymphatic Vasculature. Frontiers Cardiovasc Medicine 7: 52
Yamamizu K, Matsunaga T, Uosaki H, Fukushima H, Katayama S, Hiraoka-Kanie M, Mitani K & Yamashita J K (2010) Convergence of Notch and β-catenin signaling induces arterial fate in vascular progenitors. The Journal of Cell Biology 189: 325-338
Zeng Y, He J, Bai Z, Li Z, Gong Y, Liu C, Ni Y, Du J, Ma C, Bian L, et al (2019) Tracing the first hematopoietic stem cell generation in human embryo by single-cell RNA sequencing. Cell Res 29: 881-894
Zhou J, Xu J, Zhang L, Liu S, Ma Y, Wen X, Hao J, Li Z, Ni Y, Li X, et al (2019) Combined Single-Cell Profiling of lncRNAs and Functional Screening Reveals that H19 Is Pivotal for Embryonic Hematopoietic Stem Cell Development. Cell Stem Cell
Zovein A C, Hofmann J J, Lynch M, French W J, Turlo K A, Yang Y, Becker M S, Zanetta L, Dejana E, Gasson J C, et al (2008) Fate Tracing Reveals the Endothelial Origin of Hematopoietic Stem Cells. Cell Stem Cell 3: 625-636

1. An in vitro method of isolating hemogenic endothelial cells (HECs) or enriched populations of HECs, comprising isolating CD32+ cells from a population of cells derived from pluripotent stem cells.

2. An in vitro method according to claim 1, comprising the steps of:
i. Culturing a population of pluripotent stem cells (PSCs) in a mesoderm differentiation medium and/or hematovascular differentiation medium; and
ii. Isolating CD32+ cells from the population of cells obtained from step (i) to obtain hemogenic endothelial cells (HECs) or enriched populations of HECs.

3. The method according to claim 1 wherein said HECs are capable of generating hematopoietic cells in vitro or following transplantation into a recipient and/or are increased in numbers and percentage in cell population and/or have increased potency in differentiation and/or improved HE cellularity, as compared to culturing pluripotent stem cells having HECs not previously isolated for the presence of CD32+.

4. An in vitro method of generating a population of hematopoietic cells comprising the steps of:
i. Culturing a population of pluripotent stem cell (PSC) in a mesoderm differentiation medium and/or hematovascular differentiation medium;
ii. Isolating CD32+ cells from the population of cells obtained from step (i) to obtain hemogenic endothelial cells (HECs) or enriched populations of HECs, and
iii. culturing the obtained hemogenic endothelial cells (HECs) or enriched populations of HECs obtained from step (ii) in a medium that induces or promotes hematopoietic cells to produce a population of hematopoietic cells.

5. The method according to claim 1, wherein the pluripotent stem cells are derived embryonic stem cells or induced pluripotent stem cells (iPSCs) and/or the pluripotent stem cells (PSCs) are derived from cultures or lines or tissues.

6. The method according to claim 1, wherein said isolation step further comprises selection of cells based on expression of at least one additional endothelial marker and/or at least one marker selected from CD34, CD31, CD54, CD102, CD47, CD144, CD146, CD124, CD201, CD151, LTBR, CD39, CD147, CD105, CD143, CD309, and CD44.

7. The method according to claim 1, wherein said isolation step further comprises selection of cells that do not express at least one marker selected from CD43, CD184, CD73 and/or DLL4.

8. The method according to claim 1 wherein the cells are isolated by an immunoselection technique, optionally selected from the group consisting of flow cytometry, activated cells sorting and immune-magnetic selection, and/or by means of a specific ligand, wherein the ligand is optionally an antibody, or functional fragment thereof, by optionally using a carrier conjugated to said ligand, or by FACS, wherein the carrier is optionally a magnetic bead or wherein the carrier is loaded into a column.

9. The isolated HECs or the enriched populations of HECs obtained by the method according to claim 1.

10. A substantially pure population of CD32+ HECs or a population of hematopoietic cells derived from a substantially pure population of CD32+ HECs.

11. The isolated HECs or the enriched populations of HECs according to claim 9 or cells derived therefrom wherein the cells or the population is CD32+CD34+CD43−CD184−CD73−DLL4− and/or wherein the population comprises at least 80% or 85% or 90% of hematopoietic cells.

12. A composition comprising the isolated HECs or the enriched populations of HECs according to claim 9 or cells derived therefrom, preferably wherein said composition optionally further comprises a pharmaceutically acceptable carrier and/or a culture medium and/or a cryoprotective agent.

13. (canceled)

14. A method for the prevention and/or treatment of hematopoietic disorders and diseases, comprising administering the isolated HECs or the enriched populations of HECs of claim 9 to a patient in need thereof.

15. (canceled)

16. A kit comprising detecting means for CD32 and/or at least one of the markers as defined in claim 6.

17. The method according to claim 4, wherein the medium is a hematopoietic specification medium.

18. The method according to claim 5, wherein the pluripotent stem cells are human.

19. The population of hematopoietic cells obtained by the method of claim 4.
